(19) United States
(12) Patent Application Publication
(10) Pub. No.: US 2009/0099199 A1
(43) Pub. Date: Apr. 16, 2009

(54) ORGANIC COMPOUNDS

(76) Inventors: Joachim Nozulak, Heitersheim (DE); David Orain, Hesingue (FR); Simona Cotesta, Basel (CH)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/251,627

(22) Filed: Oct. 15, 2008

(30) Foreign Application Priority Data
Oct. 16, 2007 (EP) 07118557.3
Dec. 21, 2007 (EP) 07150297.5

(51) Int. Cl. C07D 413/12; A61K 31/4545; A61K 31/454; A61K 31/4523; A61K 31/496; A61K 31/495; C07D 405/12; C07D 405/14; C07D 403/10; C07D 241/04

(52) U.S. Cl. 514/253.11; 544/367; 514/254.04; 514/252.13; 544/374; 544/393; 514/255.03; 546/209; 546/214; 544/370; 546/193; 544/364; 514/318; 514/322; 514/326

(57) ABSTRACT

The invention relates to compound of the formula I

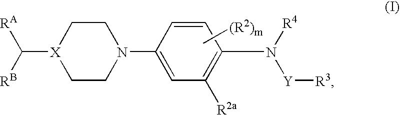

wherein the substituents are as defined in the specification; in free base form or in acid addition salt form; to its preparation, to its use as medicament and to medicaments comprising it.

The present invention relates to heterocyclic compounds, to their preparation, to their use as medicaments and to medicaments comprising them.

In a first aspect, the invention relates to a compound of the formula I

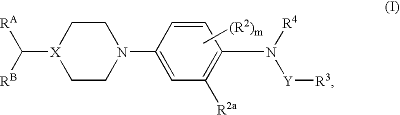

wherein

R^{A} and R^{B} independently represent an optionally substituted aryl group, an optionally substituted cycloalkyl group, an optionally substituted heteroaryl group or an optionally substituted heterocyclyl group;

X represents CH or N;

R² and R^{2a} independently represent hydrogen, a substituent different from hydrogen or a single bond attached to R⁴;

m represents an integer from 0-3;

Y represents a single bond, —C(O)—, —C(O)N(R⁵)—, —(CH₂)ₚ—, —O—, —N(R⁵)—, —O—C(O)— or —C(O)O—; or Y represents one of the following groups

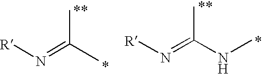

wherein R′ and R^{2a} together represent a single bond, wherein the bond marked * is attached to R³, and wherein the bond marked ** is attached to the nitrogen atom bound to the phenyl ring;

R⁵ represents hydrogen, alkyl or cycloalkyl;

p represents an integer from 1-5;

R³ represents an optionally substituted aryl group, an optionally substituted cycloalkyl group, an optionally substituted heteroaryl group, an optionally substituted heterocyclyl group or an optionally substituted alkyl group;

R⁴ represents hydrogen or a sustituent as defined for R³;

or, in case R² or R^{2a} represents a single bond attached to R⁴, R⁴ represents alkanediyl or alkenediyl, in each case optionally substituted by a substituent different from hydrogen and in each case optionally interrupted by one or more moieties selected from the group consisting of —O—, ═N— and —N(R⁵)—;

provided in case X represents N and Y represents —C(O)—, R³ does not represent substituted phenyl or substituted indolyl;

in free base form or in acid addition salt form.

The invention may be more fully appreciated by reference to the following description, including the following glossary of terms and the concluding examples. For the sake of brevity, the disclosures of the publications cited in this specification are herein incorporated by reference. As used herein, the terms “including”, “containing” and “comprising” are used herein in their open, non-limiting sense.

Any formula given herein is intended to represent compounds having structures depicted by the structural formula as well as certain variations or forms. In particular, compounds of any formula given herein may have asymmetric centers and therefore exist in different enantiomeric forms. If at least one asymmetrical carbon atom is present in a compound of the formula I, such a compound may exist in optically active form or in the form of a mixture of optical isomers, e. g. in the form of a racemic mixture. All optical isomers and their mixtures, including the racemic mixtures, are part of the present invention. Thus, any given formula given herein is intended to represent a racemate, one or more enantiomeric forms, one or more diastereomeric forms, one or more atropisomeric forms, and mixtures thereof. Furthermore, certain structures may exist as geometric isomers (i.e. cis and trans isomers), as tautomers, or as atropisomers. Additionally, any formula given herein is intended to represent hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵J respectively. Various isotopically labeled compounds of the present invention, for example those into which radioactive isotopes such as ³H, ¹³C, and ¹⁴C are incorporated. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

The acid addition salt of compounds of formula I are preferably pharmaceutically acceptable salts. Such salts are known in the field.

The following general definitions shall apply in this specification, unless otherwise specified:

Halogen (or halo) denotes fluorine, bromine, chlorine or iodine, in particular fluorine, chlorine.

The term “Alkyl” refers to a straight-chain or branched-chain alkyl group, preferably represents a straight-chain or branched-chain C₁₋₁₂alkyl, particularly preferably represents a straight-chain or branched-chain C₁₋₆alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl, iso-propyl and n-butyl and iso-butyl. Alkyl may be unsubstituted or substituted. Exemplary substituents include, but are not limited to hydroxyl, alkoxy, halogen and amino. An example of a substituted alkyl is trifluoromethyl. Cyclocalkyl may be a substituent to alkyl. An example of such a case is the moiety (alkyl)-cyclopropyl or alkandiyl-cycloproyl, e.g. —CH₂-cyclopropyl.

The term “Alkenyl” refers to a straight-chain or branched-chain alkenyl group and may be substituted or unsubstituted, preferably C₂₋₆alkenyl, for example, vinyl, allyl, 1-propenyl, isopropenyl, 2-butenyl, 2-pentenyl, 2-hexenyl, etc. and preferably represents C₂₋₄ alkenyl.

Each alkyl part of “alkoxy”, “alkoxyalkyl”, “alkoxycarbonyl”, “alkoxycarbonylalkyl” and “halogenalkyl” shall have the same meaning as described in the above-mentioned definition of “alkyl”.

The term “Alkandiyl” refers to a straight-chain or branched-chain alkandiyl group bound by two different Carbon atoms to the moiety, it preferably represents a straight-chain or branched-chain C₁₋₁₂ alkandiyl, particularly preferably represents a straight-chain or branched-chain C₁₋₆ alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

The term “Alkendiyl” refers to a straight-chain or branched-chain alkendiyl group bound by two different Carbon atoms to the molecule, it preferably represents a straight-chain or branched-chain C₂₋₆ alkandiyl; for example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—, with particular preference given to —CH═CH—CH₂—, —CH═CH—CH═CH—. Alkendiyl may be substituted or unsubstituted

The term “cycloalkyl” refers to a saturated or partially saturated, monocyclic, fused polycyclic, or Spiro polycyclic, carbocycle having from 3 to 12 ring atoms per carbocycle. Illustrative examples of cycloalkyl groups include the following moieties: cyclopropyl, cyclobutyl, cyclpentyl and cylclohexyl

The term “aryl” is known in the field. Aryl is preferably naphthyl or phenyl, in particular phenyl.

The term “heterocyclyl” refers to a saturated or partly saturated ring system containing at least one hetero atom. Preferably, heterocyclyl groups consist of 3 to 11 ring atoms of which 1-3 ring atoms are hetero atoms. Heterocycles may be present as a single ring system or as bicyclic or tricyclic ring systems; preferably as single ring system or as benz-annelated ring system. Bicyclic or tricyclic ring systems may be formed by annelation of two or more rings, by a bridging atom, e.g. Oxygen, sulfur, nitrogen or by a bridging group, e.g. alkandediyl or alkenediyl. A Heterocycle may be substituted by one or more substituents selected from the group consisting of Oxo (═O), halogen, nitro, cyano, alkyl, alkandiyl, alkenediyl, alkoxy, alkoxyalkyl, alkoxycarbonyl, alkoxycarbonylalkyl, halogenalkyl, aryl, aryloxy, arylalkyl.

The term “heteroaryl” refers to an aromatic ring system containing at least one hetero atom. Preferably, heteroaryl groups consist of 3 to 11 ring atoms of which 1-3 ring atoms are hetero atoms. Heteroary, groups may be present as a single ring system or as bicyclic or tricyclic ring systems; preferably as single ring system or as benz-annelated ring system. Bicyclic or tricyclic ring systems may be formed by annelation of two or more rings. A Heterocycle may be substituted by one or more substituents selected from the group consisting of Oxo (═O), halogen, nitro, cyano, alkyl, alkandiyl, alkenediyl, alkoxy, alkoxyalkyl, alkoxycarbonyl, alkoxycarbonylalkyl, halogenalkyl, aryl, aryloxy, arylalkyl. Examples of heterocyclyl and heteroaryl groups include: pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazlolidine, isothiazole, istothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pterine, and the corresponding benz-annelated heterocycles, e.g. indole, isoindole, cumarine, cumaronecinoline, isochinoline, cinnoline.

The term “arylalkyl” refers to an aryl group bound to the molecule via an alkyl group, such as a methyl or ethyl group, preferably phenethyl or benzyl, in particular benzyl. Similarly, cycloalkylalkyl and heterocyclyl represents a cycloalkyl group bound to the molecule via an alkyl group or a heterocyclyl group bound to the molecule via an alkyl group.

Carbon containing groups, moieties or molecules contain 1 to 8, preferably 1 to 6, more preferably 1 to 4, most preferably 1 or 2, carbon atoms. Any non-cyclic carbon containing group or moiety with more than 1 carbon atom is straight-chain or branched.

Hetero atoms are atoms other than carbon and hydrogen, preferably nitrogen (N), oxygen (O) or sulfur (S).

Halogen-substituted groups and moieties, such as alkyl substituted by halogen (halogenalkyl) can be mono-, poly- or per-halogenated.

In preferred embodiments, which are preferred independently, collectively or in any combination or sub-combination, the invention relates to a compound of the formula I, in free base form or in acid addition salt form, wherein the substituents are as defined herein.

In an advantageous embodiment, the invention relates to a compound of formula IA

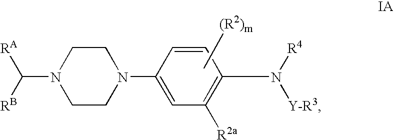

wherein R^{A}, R^{B}, R², R^{2a}, R³, R⁴ and m are as defined for a compound of formula I and wherein Y represents a single bond, —C(O)—, —C(O)N(R⁵)—, —CH₂)ₚ—, —O—, —N(R⁵)—, —O—C(O)—, —C(O)O—.

In a further advantageous embodiment, the invention relates to a compound of formula IB

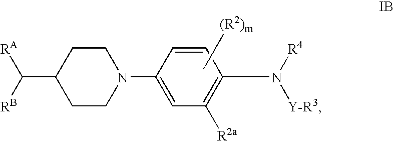

wherein R^{A}, R^{B}, R², R^{2a}, R³, R⁴ and m are as defined for a compound of formula I and wherein Y represents a single bond, —C(O)—, —C(O)N(R⁵)—, —(CH₂)ₚ—, —O—, —N(R⁵)—, —O—C(O)—, —C(O)O—.

In a further advantageous embodiment, the invention relates to a compound of formula IC

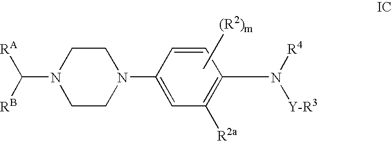

wherein R^{A}, R^{B}, R², R^{2a}, R³, R⁴ and m are as defined for a compound of formula I and wherein Y represents one of the following groups

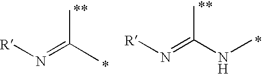

wherein R′ and R^{2a} together represent a single bond, wherein the bond marked * is attached to R³, and wherein the bond marked ** is attached to the nitrogen atom bound to the phenyl ring.

In a further advantageous embodiment, the invention relates to a compound of formula ID

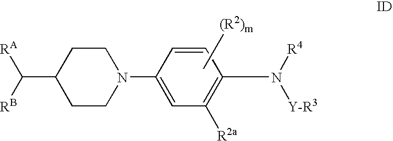

wherein R^{A}, R^{B}, R², R^{2a}, R³, R⁴ and m are as defined for a compound of formula I and wherein Y represents one of the following groups

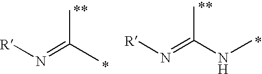

wherein R′ and R^{2a} together represent a single bond, wherein the bond marked * is attached to R³, and wherein the bond marked ** is attached to the nitrogen atom bound to the phenyl ring.

In a further advantageous embodiment, R^{2a} is hydrogen, m is 1 or 2 and R² is in the ortho-position(s) with respect to the heterocycle.

In especially preferred embodiments, the invention relates to one or more than one of the compounds of the formula I mentioned in the Examples hereinafter, in free base form or in acid addition salt form.

Y preferably represents —C(O)—, —C(O)N(R⁵)—.

R² and R^{2a} independently represent preferably hydrogen, halogen, cyano, nitro, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsufonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino, (C₁₋₈)alkoxy, (C₁₋₈)alkylamino (C₁₋₈)alkoxy, di(C₁₋₈)alkylamino (C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy.

R² and R^{2a} independently represent particular preferably a substituent selected from the group consisting of hydrogen, halogen, cyano, (C₁₋₈)alkyl, (C₁₋₈)alkyl substituted by halogen, (C₁₋₈)alkoxy, amino, (C₁₋₈)alkylamino and di(C₁₋₈)alkylamino with two identical or different (C₁₋₈)alkyl moieties.

R² and R^{2a} independently represent very particular preferably a substituent selected from the group consisting of hydrogen, fluoro, chloro, cyano, (C₁₋₄)alkyl, (C₁₋₄)alkyl substituted by fluoro.

R³ preferably represents an aryl group or a (C₃-C₈)cycloalkyl group or a heterocyclyl group with 3 to 8 ring atoms or a heteroaryl group with 3 to 8 ring atoms or a (C₁-C₈)alkyl group; wherein said aryl group, (C₃-C₈)cycloalkyl group, heteroaryl group, heterocyclyl group group is unsubstituted, mono-substituted, di-substituted or tetra-substituted, the optional substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy, —OCH₂O—, —C(═O)OCH₂—, —CH₂OC(═O)— and —CH═CHCH═CH—, the four last-mentioned optional substituents in each case being attached to two adjacent ring carbon atoms of the said moiety;

and wherein said (C₁₋₈)alkyl group is unsubstituted or mono-, di-, tri or tetra-substituted, the optional substituent(s) on the said (C₁₋₈)alkyl moiety being independently selected from the group consisting of halogen, cyano, oxo, (C₁₋₈)alkoxy, (C₁₋₈)alkoxy(C₁₋₈)alkoxy, (C₁₋₈)alkylthio, (C₁₋₈)alkylsulfinyl, (C₁₋₈)alkylsulfonyl, (C₁₋₈)alkylcarbonyloxy, (C₁₋₈)alkoxycarbonyl and (C₁₋₈)alkoxy carbonyloxy (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkyl-amino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, formyl, (C₁₋₈)-alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy.

R³ particular preferably represents an aryl group or a (C₃-C₈)cycloalkyl group or a heteroaryl group with 5 or 6 ring atoms, or a heterocyclyl group with 5 or 6 ring atoms or a (C₁-C₈)alkyl group,

which is unsubstituted or mono-, di-, tri- or tetra-substituted on the aryl group, the optional substituent(s) on said moiety being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;

which is unsubstituted or mono-, di-, tri- or tetra-substituted on the (C₃-C₈)cycloalkyl group, the optional substituent(s) on said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;

which is unsubstituted or mono-, di-, tri- or tetra-substituted on the heteroaryl group, the optional substituent(s) on the said group being independently selected from the group, consisting of halogen, cyano, nitro, amino, (C₁₋₈)alkyl, (C₁₋₈)alkyl substituted by halogen, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclylmoiety is contains 1-3 nitrogen atoms or 0-2 nitrogen and one oxygen atom;

which is unsubstituted or mono-, di-, tri- or tetra-substituted on the heterocyclyl group, the optional substituent(s) on the said group being independently selected from the group, consisting of halogen, cyano, nitro, amino, oxo, (C₁₋₈)alkyl, (C₁₋₈)alkyl substituted by halogen, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclylmoiety is contains 1-3 nitrogen atoms or 0-2 nitrogen and one oxygen atom;

which is unsubstituted in the (C₁-C₈)alkyl group.

R⁴ preferably represents hydrogen.

R⁵ preferably represents hydrogen, C₁-C₄ alkyl, C₃-C₇ cycloalkyl.

R⁵ particular preferably represents hydrogen.

m preferably represents 0,1 or 2

m particular preferably represents 0 or 1.

In one embodiment of the invention, X is CH.

In one embodiment of the invention, X is N.

R^{A} and R^{B} independently represent preferably an aryl group or a (C₃-C₈)cycloalkyl group or a heterocyclyl group with 3 to 8 ring atoms or a heteroaryl group with 3 to 8 ring atoms which is unsubstituted, mono-, di-, tri- or tetra-substituted, the optional substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkyl-amino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, formyl, (C₁₋₈)-alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy, —OCH₂O—, —C(═O)OCH₂—, —CH₂OC(═O)— and —CH═CHCH═CH—, the four last-mentioned optional substituents in each case being attached to two adjacent ring carbon atoms of the said moiety.

R^{A} and R^{B} independently represent particular preferably an aryl group or a (C₃-C₈)cycloalkyl group or a heteroaryl group with 5 or 6 ring atoms, or a heterocyclyl group with 5 or 6 ring atoms,

which is unsubstituted or mono-, di-, tri- or tetra-substituted on the aryl group, the optional substituent(s) on said moiety being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;

which is unsubstituted or mono-, di-, tri- or tetra-substituted on the (C₃-C₈)cycloalkyl group, the optional substituent(s) on said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;

which is unsubstituted or mono-, di-, tri- or tetra-substituted on the heteroaryl group, the optional substituent(s) on the said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and

whereby the heterocyclylmoiety is contains 1-3 nitrogen atoms or 0-2 nitrogen and one oxygen atom;

which is unsubstituted or mono-, di-, tri- or tetra-substituted on the heterocyclyl group, the optional substituent(s) on the said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclylmoiety is contains 1-3 nitrogen atoms or 0-2 nitrogen and one oxygen atom.

In one embodiment R^{A} represents phenyl which may be substituted by one to five substituents R¹, wherein R¹ represents a substituent different from hydrogen; preferably each R¹ independently represents a substituent selected from the group consisting of halogen, cyano, (C₁₋₈)alkyl, (C₁₋₈)alkyl substituted by halogen, (C₁₋₈)alkoxy, amino, (C₁₋₈)alkylamino and di(C₁₋₈)alkylamino with two identical or different (C₁₋₈)alkyl moieties; further preferably each R¹ independently represents a substituent selected from the group consisting of fluoro, chloro, cyano, (C₁₋₄)alkyl and (C₁₋₄)alkyl substituted by fluoro; preferably, said phenyl group is unsubstituted or monosubstituted, further preferably, said phenyl group is unsubstituted.

In one embodiment R^{A} represents pyridyl which may be substituted by one to four substituents R¹, wherein R¹ represents a substituent different from hydrogen; preferably each R¹ independently represents a substituent selected from the group consisting of halogen, cyano, (C₁₋₈)alkyl, (C₁₋₈)alkyl substituted by halogen, (C₁₋₈)alkoxy, amino, (C₁₋₈)alkylamino and di(C₁₋₈)alkylamino with two identical or different (C₁₋₈)alkyl moieties; further preferably each R¹ independently represents a substituent selected from the group consisting of fluoro, chloro, cyano, (C₁₋₄)alkyl and (C₁₋₄)alkyl substituted by fluoro; preferably, said pyridyl group is unsubstituted or monosubstituted, further preferably, said pyridyl group is unsubstituted.

In one embodiment R^{B} represents a heteroaryl group with 5 or 6 ring atoms, or a heterocyclyl group with 5 or 6 ring atoms,

which is unsubstituted or mono-, di, tri- or tetra-substituted on the heteroaryl group, the optional substituent(s) on the said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclylmoiety is contains 1-3 nitrogen atoms or 0-2 nitrogen and one oxygen atom;

which is unsubstituted or mono-, di-, tri- or tetra-substituted on the heterocyclyl group, the optional substituent(s) on the said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclylmoiety is contains 1-3 nitrogen atoms or 0-2 nitrogen and one oxygen atom.

In one embodiment R^{B} represents unsubstituted pyridyl.

Further preferred are compounds of the formula IAA

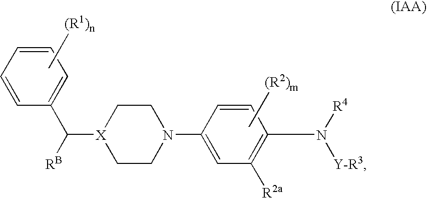

wherein

R^{B} represents an optionally substituted aryl group, an optionally substituted cycloalkyl group, an optionally substituted heteroaryl group, an optionally substituted heterocyclyl group;

R¹ represents a substituent different from hydrogen;

n represents an integer from 0-5;

X represents CH or N;

R² and R^{2a} independently represent hydrogen or a substituent as defined for R¹; or a single bond attached to R⁴;

m represents an integer from 0-3;

Y represents a single bond, —C(O)—, —C(O)N(R⁵)—, —(CH₂)ₚ—, —O—, —N(R⁵)—, —O—C(O)—, —C(O)O—;

or Y represents one of the following groups

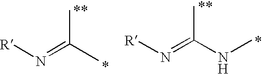

wherein R′ and R^{2a} together represent a single bond, wherein the bond marked * is attached to R³, and wherein the bond marked ** is attached to the nitrogen atom bound to the phenyl ring;

R⁵ represents hydrogen, alkyl, cycloalkyl;

p represents an integer from 0-5;

R³ represents an optionally substituted aryl group, an optionally substituted cycloalkyl group, an optionally substituted heteroaryl group, an optionally substituted heterocyclyl group; an optionally substituted alkyl group;

R⁴ represents hydrogen or a sustituent as defined for R³;

or, in case R² or R^{2a} represents a single bond attached to R⁴, R⁴ represents alkanediyl or alkenediyl, in each case optionally substituted by a substituent as defined for R¹ and in each case optionally interrupted by one or more moieties selected from the group consisting of —O—, ═N—; —N(R⁵)—;

provided in case X represents N and Y represents —C(O)—, R³ does not represent substituted phenyl or substituted indolyl;

in free base form or in acid addition salt form.

R^{B} preferably represents an aryl group or a (C₃-C₈)cycloalkyl group or a heterocyclyl group with 3 to 8 ring atoms or a heteroaryl group with 3 to 8 ring atoms which is unsubstituted, mono-, di-, tri- or tetra-substituted, the optional substituent(s) being independently selected from the group consisting of halogen, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, cyano, nitro, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino(C₁₋₈)alkoxy, (C₁₋₈)alkylamino(C₁₋₈)alkoxy, di(C₁₋₈)alkylamino(C₁₋₈)alkoxy with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl, (C₁₋₈)alkoxycarbonyl(C₁₋₈)alkoxy, —OCH₂O—, —C(═O)OCH₂—, —CH₂OC(═O)— and —CH═CHCH═CH—, the four last-mentioned optional substituents in each case being attached to two adjacent ring carbon atoms of the said moiety.

R^{B} particular preferably represents an aryl group or a (C₃-C₈)cycloalkyl group or a heteroaryl group with 5 or 6 ring atoms, or a heterocyclyl group with 5 or 6 ring atoms, which is unsubstituted or mono-, di-, tri- or tetra-substituted on the aryl group, the optional substituent(s) on said moiety being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;

which is unsubstituted or mono-, di-, tri- or tetra-substituted on the (C₃-C₈)cycloalkyl group, the optional substituent(s) on said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy;

which is unsubstituted or mono-, di-, tri- or tetra-substituted on the heteroaryl group, the optional substituent(s) on the said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclylmoiety is contains 1-3 nitrogen atoms or 0-2 nitrogen and one oxygen atom;

which is unsubstituted or mono-, di-, tri- or tetra-substituted on the heterocyclyl group, the optional substituent(s) on the said group being independently selected from the group, consisting of halogen, cyano, (C₁₋₈)alkyl, (C₁₋₈)alkyl substituted by halogen, nitro, (C₁₋₈)alkoxy, (C₁₋₈)alkoxy substituted by halogen, (C₁₋₈)alkylthio, formyloxy, (C₁₋₈)alkylcarbonyloxy; and whereby the heterocyclylmoiety is contains 1-3 nitrogen atoms or 0-2 nitrogen and one oxygen atom.

R¹ preferably and independently represents halogen, cyano, nitro, (C₁₋₈)alkyl, (C₁₋₈)alkyl substituted by halogen, (C₃₋₈)cycloalkyl, (C₃₋₈)cycloalkyl(C₁₋₈)alkyl, (C₃₋₈)cycloalkoxy, (C₃₋₈)cycloalkoxy(C₁₋₈)alkyl, (C₃₋₈)cycloalkyl(C₁₋₈)alkoxy, (C₃₋₈)cycloalkoxy(C₁₋₈)alkoxy, aryl, aryl(C₁₋₈)alkyl, aryloxy, aryloxy(C₁₋₈)alkyl, aryl(C₁₋₈)alkoxy, aryloxy(C₁₋₈)alkoxy, carboxy, carbamyl, hydroxy, (C₁₋₈)alkoxy, (C₁₋₈)alkoxy(C₁₋₈)alkoxy, (C₁₋₈)alkoxy substituted by halogen, (C₁₋₈)alkoxy(C₁₋₈)alkyl, (C₁₋₈)alkylthio, (C₁₋₈)alkylthio(C₁₋₈)alkyl, (C₁₋₈)alkylsulfinyl, (C₁₋₈)alkylsulfinyl(C₁₋₈)alkyl, (C₁₋₈)alkylsulfonyl, (C₁₋₈)alkylsulfonyl(C₁₋₈)alkyl, amino, (C₁₋₈)alkylamino, di(C₁₋₈)alkylamino with two identical or different (C₁₋₈)alkyl moieties, amino(C₁₋₈)alkyl, (C₁₋₈)alkylamino(C₁₋₈)alkyl, di(C₁₋₈)alkylamino(C₁₋₈)alkyl with two identical or different (C₁₋₈)alkyl moieties in the di(C₁₋₈)alkylamino moiety, amino, (C₁₋₈)alkoxy, (C₁₋₈)alkylamino (C₁₋₈)alkoxy, di(C₁₋₈)alkylamino (C₁₋₈)alkoxy with two identical or different (C₁₋₈)alkyl moieties, aminosulfonyl, (C₁₋₈)alkylaminosulfonyl, di(C₁₋₈)alkylaminosulfonyl with two identical or different (C₁₋₈)alkyl moieties, formyl, (C₁₋₈)alkylcarbonyl, formyloxy, (C₁₋₈)alkylcarbonyloxy, formyl(C₁₋₈)alkyl, (C₁₋₈)alkylcarbonyl(C₁₋₈)alkyl, formyl(C₁₋₈)alkoxy, (C₁₋₈)alkylcarbonyl(C₁₋₈)alkoxy, (C₁₋₈)alkoxycarbonyl, (C₁₋₈)alkoxycarbonyloxy, (C₁₋₈)alkoxycarbonyl(C₁₋₈)alkyl and (C₁₋₈)alkoxycarbonyl(C₁₋₈)alkoxy.

R¹ independently and particular preferably represents a substituent selected from the group consisting of halogen, cyano, (C₁₋₈)alkyl, (C₁₋₈)alkyl substituted by halogen, (C₁₋₈)alkoxy, amino, (C₁₋₈)alkylamino and di(C₁₋₈)alkylamino with two identical or different (C₁₋₈)alkyl moieties;

R¹ independently and very particular preferably represents a substituent selected from the group consisting of fluoro, chloro, cyano, (C₁₋₄)alkyl, (C₁₋₄)alkyl substituted by fluoro.

n preferably represents 0,1 or 2

n particular preferably represents 0.

Further preferred substituents or substituent combinations for compounds of formula IAA are as described for compounds of formula I.

The invention further relates to pharmaceutically acceptable prodrugs and pharmaceutically acceptable metabolites of a compound of formula (I).

In preferred embodiments, compounds of the present invention are selected from the group consisting of

1-[4-(4-Benzhydryl-piperazin-1-yl)-3-cyano-phenyl]-3-(3,5-dimethyl-isoxazol-4-yl)-urea;

Tetrahydro-furan-3-carboxylic acid [4-(4-benzhydryl-piperazin-1-yl)-3-cyano-phenyl]-amide;

N-[4-(4-Benzhydryl-piperazin-1-yl)-3-cyano-phenyl]-2-ethyl-butyramide;

1-[4-(4-Benzhydryl-piperazin-1-yl)-3-fluoro-phenyl]-3-(3,5-dimethyl-isoxazol-4-yl)-urea;

Tetrahydro-furan-3-carboxylic acid [4-(4-benzhydryl-piperazin-1-yl)-3-cyano-phenyl]-amide;

N-[4-(4-Benzhydryl-piperazin-1-yl)-3-fluoro-phenyl]-2-ethyl-butyramide;

1-(4-{4-[Bis-(4-fluoro-phenyl)-methyl]-piperazin-1-yl}-3-cyano-phenyl)-3-(3,5-dimethyl-isoxazol-4-yl)-urea;

1-(4-{4-[Bis-(4-fluoro-phenyl)-methyl]-piperazin-1-yl}-3-fluoro-phenyl)-3-(3,5-dimethyl-isoxazol-4-yl)-urea;

3,5-Dimethyl-isoxazole-4-carboxylic acid [4-(4-benzhydryl-piperazin-1-yl)-3-fluoro-phenyl]-amide;

N-[4-(4-Benzhydryl-piperazin-1-yl)-3-fluoro-phenyl]-2-methyl-nicotinamide;

1-Methyl-1H-pyrrole-2-carboxylic acid [4-(4-benzhydryl-piperazin-1-yl)-3-fluoro-phenyl]-amide;

2-Methyl-2H-pyrazole-3-carboxylic acid [4-(4-benzhydryl-piperazin-1-yl)-3-fluoro-phenyl]-amide;

1-[4-(4-Benzhydryl-piperidin-1-yl)-3-cyano-phenyl]-3-(3,5-dimethyl-isoxazol-4-yl)-urea;

Tetrahydro-furan-3-carboxylic acid [4-(4-benzhydryl-piperidin-1-yl)-3-cyano-phenyl]-amide;

N-[4-(4-Benzhydryl-piperidin-1-yl)-3-cyano-phenyl]-2-ethyl-butyramide;

1-[4-(4-Benzhydryl-piperidin-1-yl)-3-fluoro-phenyl]-3-(3,5-dimethyl-isoxazol-4-yl)-urea;

Tetrahydro-furan-3-carboxylic acid [4-(4-benzhydryl-piperidin-1-yl)-3-fluoro-phenyl]-amide;

N-[4-(4-Benzhydryl-piperidin-1-yl)-3-fluoro-phenyl]-2-ethyl-butyramide;

5-(4-Benzhydryl-piperazin-1-yl)-2-(1-ethyl-propyl)4-fluoro-1H-benzoimidazole;

5-(4-Benzhydryl-piperazin-1-yl)-6-fluoro-2-(tetrahydro-furan-3-yl)-1H-benzoimidazole;

5-(4-Benzhydryl-piperazin-1-yl)-2-(3,5-dimethyl-isoxazol-4-yl)-6-fluoro-1H-benzoimidazole;

[5-(4-Benzhydryl-piperazin-1-yl)-6-fluoro-1H-benzoimidazol-2-yl]-(3,5-dimethyl-isoxazol-4-yl)-amine;

5-(4-Benzhydryl-piperazin-1-yl)-6-fluoro-1H-benzoimidazol-2-yl]-(1-ethyl-propyl)-amine;

N-4-[4-(Di-pyridin-2-yl-methyl)-piperidin-1-yl]-3-fluoro-phenyl)-2-ethyl-butyramide; and

2-Ethyl-N-{3-fluoro-4-[4-((R)-pyridin-2-yl-tetrahydro-furan-2-yl-methyl)-piperazin-1-yl]-phenyl}-butyramide;

and pharmaceutically acceptable salts, prodrugs and metabolites thereof.

In a further aspect, the invention relates to processes for the preparation of compounds of the formula I and their salts.

Compounds of formula IA are obtainable according to Process A which is summarized by the following scheme.

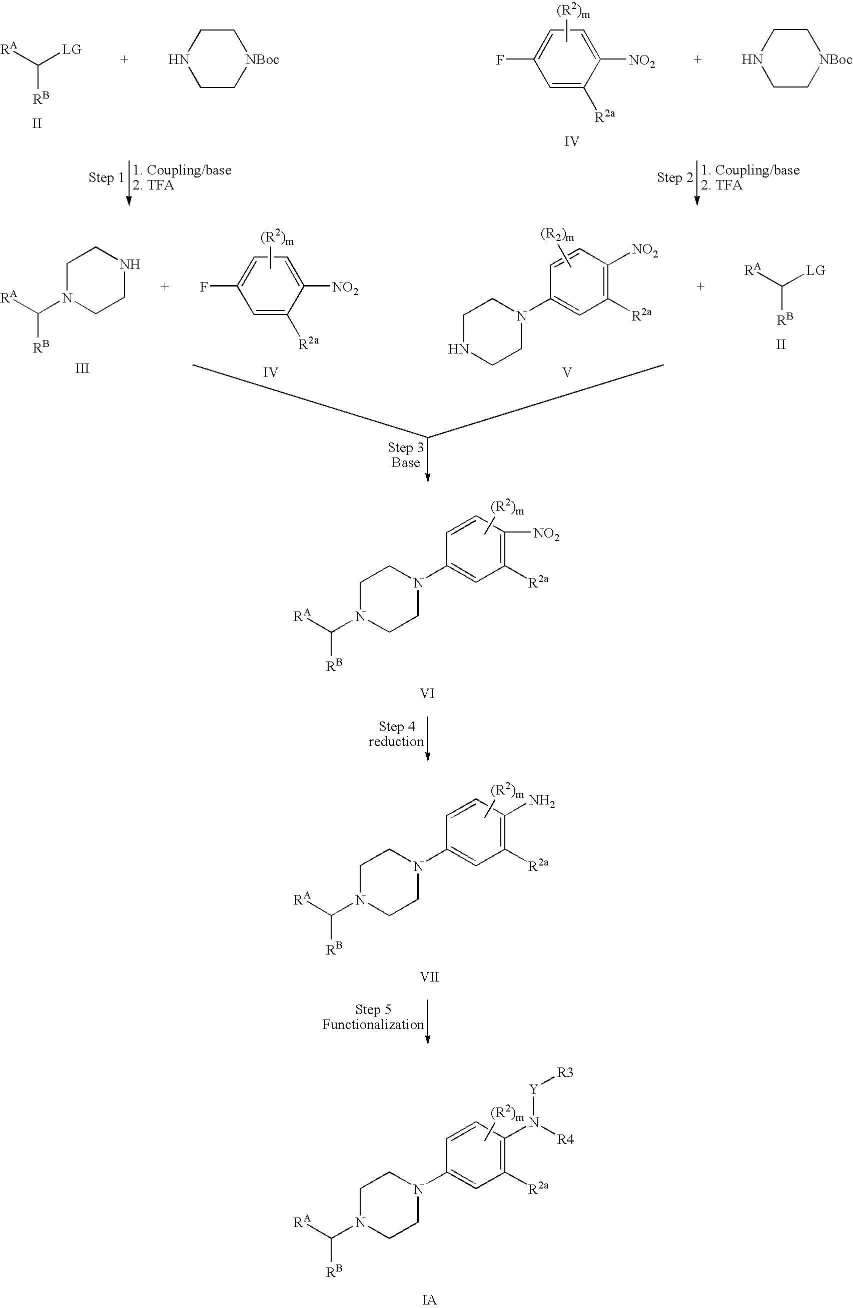

The process steps are described in more detail below:

Step 1: A compound of formula (III) is obtainable by reacting a compound of formula (II) where R represented any cycle and LG a leaving group such as bromo or chloro with Boc-piperazine in the presence of a suitable base e.g. triethylamine or potassium carbonate, optionally in the presence of a solvent, e.g. dioxane, DMF followed by treatment with TFA in suitable solvent, e.g DCM or dioxane.

Step 2: A compound of formula (V) is obtainable by reacting a compound of formula (IV) with Boc-piperazine in the presence of a suitable base e.g. triethylamine or potassium carbonate, optionally in the presence of a solvent, e.g. dioxane, DMF followed by treatment with TFA in suitable solvent, e.g DCM or dioxane.

Step 3: A compound of formula (VI) is obtainable by reacting a compound of formula (V) with a compound of formula (II) where R represented any cycle and LG a leaving group such as bromo or chloro or by reacting a compound of formula (III) with a compound of formula (IV) in the presence of a suitable base e.g. triethylamine or potassium carbonate, optionally in the presence of a solvent, e.g. dioxane, DMF.

Step 4: A compound of formula (VII) is obtainable by reacting a compound of formula (VI) with a reductive agent, e.g tin chloride, Ra/Ni, iron powder in suitable solvent, e.g EtOH, AcOH or EtOAc.

Step 5: A compound of formula (I) is obtainable by reacting a compound of formula (VII) with alkylating or arylating agent, e.g Mel, 2-chloropyridine, or an aldehyde or an acid or an isocyanate.

Compounds of formula IB, are obtainable according to Process B which is summarized by the following scheme.

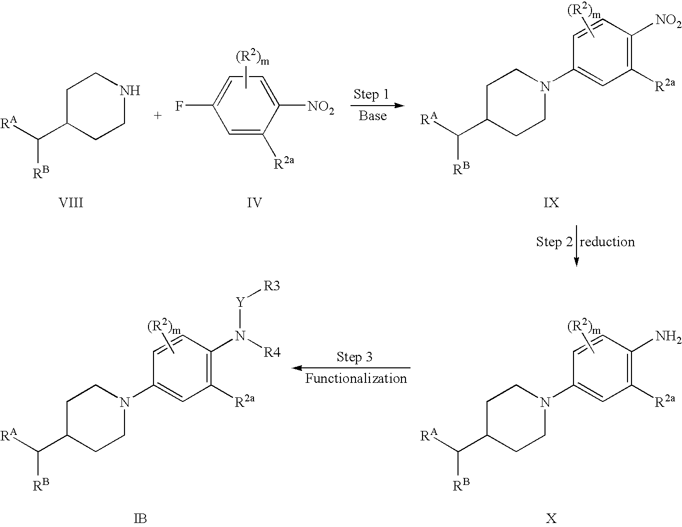

The process steps are described in more detail below:

Step 1: A compound of formula (IX) is obtainable by reacting a compound of formula (IV) with a compound of formula (VIII) wherein the substituents are as defined in formula I, in the presence of a suitable base e.g. triethylamine or potassium carbonate, optionally in the presence of a solvent, e.g. dioxane, DMF.

Step 2: A compound of formula (X) is obtainable by reacting a compound of formula (IX) with a reductive agent, e.g tin chloride, Ra/Ni, iron powder in suitable solvent, e.g EtOH, AcOH or EtOAc.

Step 3: A compound of formula (IB) is obtainable by reacting a compound of formula (X) with alkylating or arylating agent, e.g Mel, 2-chloropyridine, or an aldehyde or an acid or an isocyanate.

Compounds of formula IC and ID are obtainable according to Process C which is summarized by the following scheme.

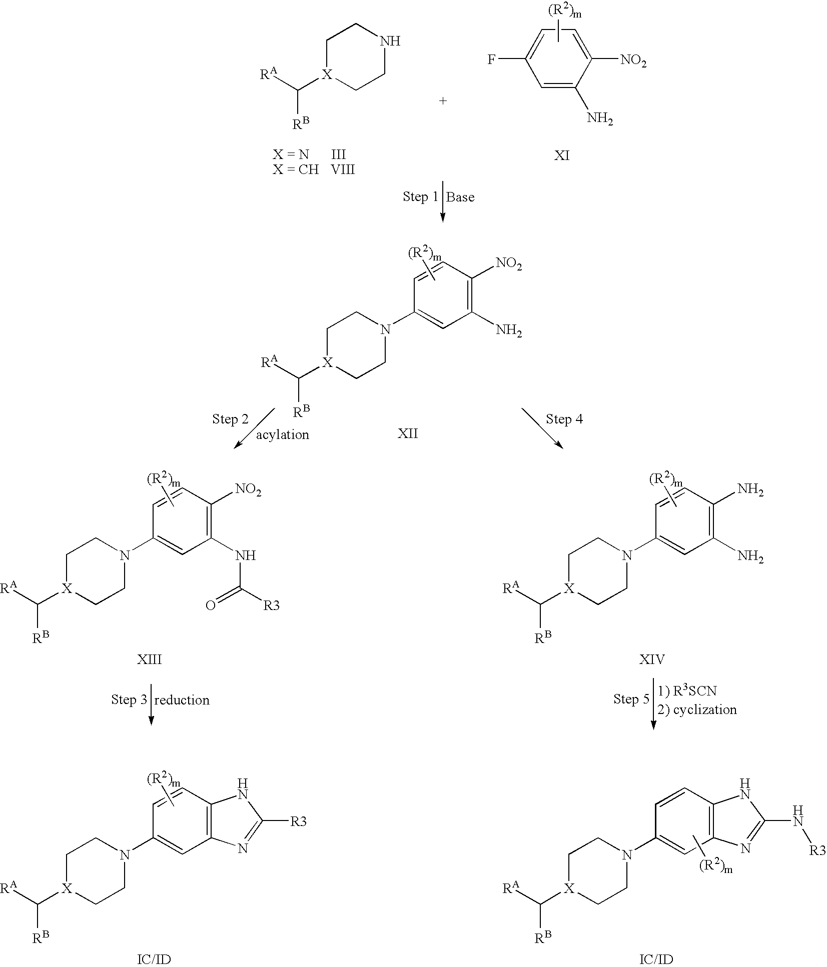

The process steps are described in more detail below:

Step 1: A compound of formula (XII) is obtainable by reacting a compound of formula (XI) with a compound of formula (III) or (VIII), wherein the substituents are as defined in formula I, in the presence of a suitable base e.g. triethylamine or potassium carbonate, optionally in the presence of a solvent, e.g. dioxane, DMF.

Step 2: A compound of formula (XIII) is obtainable by reacting a compound of formula (XII), wherein the substituents are as defined in formula I, with a carboxylic acid or carboxylic acid chloride in the presence of a suitable activating agent e.g. HOBt or DCC, or a base e.g. triethylamine or potassium carbonate, optionally in the presence of a solvent, e.g. dioxane, DMF.

Step 3: A compound of formula (IC, ID) is obtainable by reacting a compound of formula (XIII), wherein the substituents are as defined in formula I, with a reductive agent, e.g tin chloride, Ra/Ni, iron powder in suitable solvent, e.g EtOH, AcOH or EtOAc.

Step 4: A compound of formula (XIV) is obtainable by reacting a compound of formula (XII), wherein the substituents are as defined in formula I, with a reductive agent, e.g tin chloride, Ra/Ni, iron powder in suitable solvent, e.g EtOH, AcOH or EtOAc.

Step 5: A compound of formula (IC, ID) is obtainable by reacting a compound of formula (XIV), wherein the substituents are as defined in formula I, with an isothiocyanate derivative in the presence of a suitable base e.g. triethylamine or potassium carbonate, optionally in the presence of a solvent, e.g. dioxane, DMF followed by cyclisation mediated by HgO/S.

Thus, the invention also relates to processes for manufacturing a compound of formula I according to the described methods above.

All reactions for manufacturing compounds of formula I can be effected according to conventional methods, for example as described in the Examples. The working-up of the reaction mixtures and the purification of the compounds thus obtainable may be carried out in accordance with known procedures. Acid addition salts may be produced from the free bases in known manner, and vice-versa.

Compounds of the formula I can also be prepared by further conventional processes, e. g. as described in the Examples, which processes are further aspects of the invention.

The starting materials of the formulae II, III, IV, VIII and XI are known or may be prepared according to conventional procedures starting from known compounds, for example as described in the Examples. Some of the intermediates of formulae V, VI, VII, IX, X, XII, XIII and XIV are novel and subject of the present invention.

It was further found that compounds of the formula I′

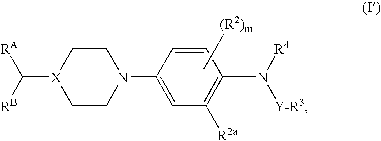

wherein

R^{A} and R^{B} independently represent an optionally substituted aryl group, an optionally substituted cycloalkyl group, an optionally substituted heteroaryl group, an optionally substituted heterocyclyl group;

X represents CH or N;

R² and R^{2a} independently represent hydrogen or a substituent as defined for R¹; or a single bond attached to R⁴;

m represents an integer from 0-3;

Y represents a single bond, —C(O)—, —C(O)N(R⁵)—, —(CH₂)ₚ—, —O—, —N(R⁵)—, —O—C(O)—, —C(O)O—;

or Y represents one of the following groups

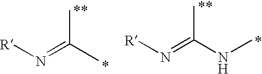

wherein R′ and R^{2a} together represent a single bond, wherein the bond marked * is attached to R³, and wherein the bond marked ** is attached to the nitrogen atom bound to the phenyl ring;

R⁵ represents hydrogen, alkyl, cycloalkyl;

p represents an integer from 0-5;

R³ represents an optionally substituted aryl group, an optionally substituted cycloalkyl group, an optionally substituted heteroaryl group, an optionally substituted heterocyclyl group; an optionally substituted alkyl group;

R⁴ represents hydrogen or a sustituent as defined for R³;

or, in case R² or R^{2a} represents a single bond attached to R⁴, R⁴ represents alkanediyl or alkenediyl, in each case optionally substituted by a substituent as defined for R¹ and in each case optionally interrupted by one or more moieties selected from the group consisting of —O—, ═N—; —N(R⁵)—;

provided in case X represents N and Y represents —C(O)—, R³ does not represent substituted phenyl or substituted indolyl;

and their pharmaceutically acceptable acid addition salts, hereinafter also referred to as “agents of the invention”, exhibit valuable pharmacological properties, when tested in vitro and in animals, and are, therefore, useful as active ingredients in medicaments. Agents of the invention have good efficacy as selective ligands for NPY Y2 receptors, showing desirable NPY Y2 receptor modulating activities at various receptor subtypes, and, moreover, may possess interesting pharmacokinetic properties, e. g. improved oral bioavailability or enhanced metabolic stability.

Thus, in a further aspect, the invention provides a method for the treatment, prevention or delay of progression of a condition, disease or disorder, that can be modulated or is mediated by NPY Y2 receptor comprising administering to a subject in need thereof a therapeutically effective amount of a compound of the formula I or I′, in free form or in pharmaceutically acceptable salt form.

The invention also provides the use of a compound of the formula I or I′, in free form or in pharmaceutically acceptable salt form, for the manufacture of a medicament for the treatment, prevention or delay of progression of a condition, disease or disorder, that can be modulated or is mediated by NPY Y2 receptors.

The invention therefore relates to novel non-peptidic NPY Y2 receptor modulators, in particular inhibitors, useful in treating or preventing disorder: anxiety disorders and depression; injured mammalian nerve tissue; a condition responsive to treatment through administration of a neurotrophic factor; a neurological disorder; bone loss; substance related disorders; sleep/wake disorders; cardiovascular disease; metabolic disorders such as obesity; or an obesity-related disorder. Compounds of the invention are also useful in modulating endocrine functions; particularly endocrine functions controlled by the pituitary and hypothalamic glands, and maybe used to treat inovulation and infertility.

Neuropeptide Y (NPY) is a highly conserved 36 amino acid peptide that belongs to the pancreatic polypeptide (PP) family and was first isolated from mammalian brain in 1982 (Tatemoto et al., 1982 Nature 1982, 296, 659). NPY sequences from a number of animal species have been elucidated and all show a high degree of amino acid homology to the human protein (see Larhammar, D. In “The Biology of Neuropeptide Y and Related Peptides”, Colmers, W. F. and Wahlestedt, C. Eds., Humana Press, Totowa, N.J. 1993). NPY is one of the most abundant neuropeptides in the mammalian central (CNS) and peripheral nervous systems (PNS) and controls a wide spectrum of basic physiological functions. NPY strongly stimulates food intake, affects blood pressure and cardiovascular function through its vasoconstricting properties, induces anxiolysis, affects circadian rhythms and controls certain aspects of endocrine hypothalamic and pituitary functions (Heilig and Widerlöv, 1995; Thorsell and Heilig, 2002). Furthermore, evidence has accumulated supporting a role of NPY in memory processing, drug and alcohol abuse, pain, and epilepsy (Silva et al., 2002). Among the potential physiological properties of NPY, its orexigenic effect has been most widely studied and was first suggested by the demonstration that NPY potently stimulates food intake following acute injection into the brain ventricles or into specific hypothalamic sites such as the paraventricular nucleus in rats (Levens and Della-Zuana, 2003).

In mammals, the NPY gene is expressed in neurons where NPY itself is mainly found. In the brain, NPY is expressed at high levels in hypothalamic areas, nucleus accumbens, septum, and periaqueductal gray matter. Moderate expression levels of NPY are found in amygdala, hippocampus, thalamus, and basal ganglia. NPY expression is almost absent in the pons and cerebellum. In the forebrain, the interneurons are the predominant NPY-immunoreactive neurons (Thorsell and Heilig, 2002).

At the cellular level, NPY exerts its biological effects through an interaction with a portfolio of receptors. Presently, five receptors for NPY have been characterized based upon binding profile, pharmacological characterization, and cDNA sequence—Y1, Y2, Y4, Y5, and Y6 (Kaga, T. et al. Peptides 2001, 22, 501-506; Wahlestedt, C. et al. Ann. N.Y. Acad. Sd. 1990, 611, 7; Larhammar, D. et al. J. BioJ. Chem. 1992, 267, 10935; Wahlestedt, C. et al. Regul Pept. 1986, 13, 307; Fuhlendorff, J. U. et al. Proc. Natl. Acad. Sd. U.S.A. 1990, 87, 182; Grundemar, L. et al. J. Pharmacol. Exp. Ther. 1991, 258, 633; Laburthe, M. et al. Endocrinology 1986, 118, 1910; Castan, I. et al. Endocrinology 1992, 131, 1970; Gerald, C. et al. Nature 1996, 382, 168; Weinberg, D. H. et al. J. Biol. Chem. 1996, 271, 16435; Gehlert, D. et al. Curr. Pharm. Des. 1995, 1, 295; Lundberg, J. M. et al. Trends Pharmacol. Sci. 1996, 17, 301). The NPY Y₆ receptor is not functional in humans while NPY does not bind to human Y₄ receptors. A Y3 has not been cloned but only pharmacologically characterized (Michel et al., 1998; Silva et al., 2002). All NPY receptors belong to the family of the so-called G-protein coupled receptors (GPCRs). Among the typical signaling responses of NPY receptors are inhibition of adenylyl cyclase and increase of intracellular calcium concentration through IP3-dependent mobilization of intracellular calcium stores or via action on calcium channels.

Binding of NPY to its receptors can elicit a variety of pharmacological and biological effects in vitro and in vivo. A host of preclinical evidence has accumulated supporting a role of NPY in the control of anxiety-like behavior. For example, when administered to the brain of live animals (intracerebroventricularly (icv) or into the amygdala), NPY produced anxiolytic-like effects in established animal models of anxiety such as the elevated plus-maze, Vogel punished drinking, Geller-Seifter's bar-pressing conflict paradigms, and fear-potentiated startle (Broqua et al., 1995; Thorsell and Heilig, 2002; Heilig, M. et al. Psychopharmacology 1989, 98, 524; Heilig, M. et al. Regul. Pept. 1992, 41, 61; Heilig, M, et al. Neuropsychopharmacology 1993, 8,357). In humans intravenous administration of NPY has been shown to inhibit hypothalamic-pituitary-adrenal (HPA) axis activity, promote sleep and modulate REM sleep (Antonijevic et al., 2000). Thus, compounds that mimic NPY are postulated to be useful for the treatment of anxiety disorders and sleep disorders.

NPY immunoreactivity is significantly decreased in the cerebrospinal fluid (CSF) of patients with major depression and those of suicide victims (Widdowson, P. S. et al. J. Neurochem. 1992, 59, 73), and rats treated with tricyclic antidepressants displayed significant increases of NPY levels relative to vehicle-treated animals (Heilig, M. et al). Eur. J. Pharmaco). 1988, 147, 465). These findings suggest that an inadequate NPY response may play a role in the pathophysiology of depression, and that compounds that regulate and potentiate the NPY-ergic system may be useful for the treatment of depression.

It is well accepted that the anxiolytic properties of NPY are mediated through its postsynaptic Y1 receptors, whereas presynaptic Y2 receptors negatively modulate the release of NPY and that of other neurotransmitters (such as GABA, glutamate and others). Consequently, Y2 receptor blockade may lead to enhanced GABA-ergic and NPY-ergic effects and thus Y2 receptor antagonists may prove useful in the treatment of depression and anxiety.

NPY improved memory and performance scores in animal models of learning (Flood, J. F. etal. Brain Res. 1987, 421, 280) and therefore may serve as a cognition enhancer for the treatment of neurodegenerative diseases such as Alzheimer's Disease (AD) as well as AIDS-related and senile dementia.

Elevated plasma levels of NPY were present in animals and humans experiencing episodes of high sympathetic nerve activity such as surgery, newborn delivery, and hemorrhage (Morris, M. J. et. al. J. Auton. Nerv. Syst 1986, 17, 143). Thus, chemical substances that alter the NPY-ergic system may be useful for alleviating migraine, pain, and the condition of stress.

NPY also mediates endocrine functions such as the release of luteinizing hormone (LH) in rodents (Kalra, S. P. et. al. Front. Neuroendrocrinol. 1992, 13, 1). Since LH is vital for mammalian ovulation, a compound that mimics the action of NPY could be useful for the treatment of infertility, particularly in women with so-called luteal phase defects.

Release of NPY and activation of NPY Y2 receptors stimulates fat angiogenesis and the proliferation and angiogenesis of new adipocytes, resulting in abdominal obesity and a metabolic syndrome-like condition in mice. While NPY was shown to stimulate mouse and human fat growth, local abdominal fat-delivered NPY Y2 receptor antagonist decreased adipose tissue weight and volume in both obese and lean mice by 50%. The lipolytic effect of Y2 receptor blockade was accompanied by decreased vascularity and increased apoptosis in the abdominal fat pads (Kuo, L. E. et al. Nat. Med. 2007 13(7), 803). Thus, compounds that block NPY Y2 receptors may be useful for the treatment of obesity and metabolic disorders. Furthermore, local administration of NPY Y2 receptor antagonists might be useful for nonsurgical localized removal of fat (pharmacological lipolysis).

Y2 receptor knockout mice displayed reduced body weight despite an increase in food intake, possibly due to the lack of the feedback inhibition of the postprandially released anorectic peptide PYY3-36 (Batterham, R. L. et al. Nature 2002, 418, 650-654). The Y2 receptor knockout mice also showed a significant increase in bone mineral density (Baldock, P. A. J. Clin. Invest. 2002, 109, 915-921). Furthermore, hypothalamic specific deletion of Y2 receptors in adult Y2 receptor floxed mice was reported to produce an increase in bone mineral density. Thus NPY Y2 antagonists may be useful for the prevention and treatment of osteoporosis.

A direct link between NPY signaling and regulation of ethanol consumption was suggested by the demonstration that NPY overexpression in mice reduced ethanol self-administration, whereas NPY knockout increased ethanol self-administration (Thiele et al. Nature 1998, 396, 366-369). Studies have also indicates that the Y2 receptor is involved in the neurobiological responses to ethanol and other drugs of abuse. Thiele and coworkers (Neuropeptides, 2004, 38(4), 235-243; Peptides 2004, 25(6), 975-983) described low ethanol consumption of Y2 receptor knockout mice, as well as their increased voluntary water consumption. Recently, it has been demonstrated that icv administration of BIIE0246, a selective NPY Y2 antagonist, dose dependently reduced ethanol self-administration in rats (Thorsell et al. Neurosci. Lett. 2002, 332, 14). Therefore, NPY Y2 receptor antagonists may be useful for the treatment of alcohol and drug abuse.

Additionally, NPY Y2 antagonists have been suggested for the prevention of cardiovascular disease, for example, sudden death due to cardiac arrhythmias, post-myocardial infarction, or heart failure (See: Intl. Pat. Appl. Publ. WO 02/083137, Oct. 24, 2002).

The invention includes also pharmaceutically acceptable salts of the compounds represented by Formula I or I′. Pharmaceutically acceptable salts of the above-described specific compounds are especially preferred. See, e.g., S. M. Berge, etaL, “Pharmaceutical Salts”, J. Pharm. Sd., 1977, 66:1-19, and Handbook of Pharmaceutical Salts, Propertions, Selection, and Use; Stahl, R H., Wermuth, C. G., Eds.; Wiley-VCH and VHCA: Zurich, 2002. However, salts of acids and bases that are non-pharmaceutically acceptable may also find use, for example, in the preparation or purification of a pharmaceutically acceptable compound. All salts, whether pharmaceutically acceptable or not are included within the ambit of the present invention. A “pharmaceutically acceptable salt” is intended to mean a salt of a free acid or base of a compound represented by Formula I or I′ that is not toxic, biologically intolerable, or otherwise biologically undesirable. Preferred pharmaceutically acceptable salts are those that are pharmacologically effective and suitable for contact with the tissues of patients without undue toxicity, irritation, or allergic response.

The invention also relates to treatment methods employing pharmaceutically acceptable prodrugs of the compounds of Formula I or I′. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I) or (H)) A “pharmaceutically acceptable prodrug” is a prodrug that is not toxic, biologically intolerable, or otherwise biologically unsuitable for administration to the subject. Conventional procedures for the selection and preparation of suitable prodrug derivatives are described, for example, in “Design of Prodrugs”, ed. H. Bundgaard, Elsevier, 1985. Exemplary prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula I or I′.

Pharmaceutically active metabolites may also be used in the methods of the invention. A “pharmaceutically active metabolite” means a pharmacologically active product of metabolism in the body of a compound of Formula I or I′ or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sd. 1997, 86(7), 765-767; Bagshawe, Drug 0ev. Rs. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

The compounds of Formula I or I′ and their pharmaceutically acceptable salts, pharmaceutically acceptable prodrugs, and pharmaceutically active metabolites of the present invention are useful as NPY Y2 modulators, in particular inhibitors, in the methods of the invention. The agents may be used in the inventive methods for the treatment or prevention of medical conditions, diseases, or disorders mediated through inhibition or modulation of NPY Y2, such as those described herein. Compounds of the invention are potent, non-peptidic, low molecular weight, selective NPY Y2 inhibitors and are useful in treating or preventing: anxiolytic disorders and depression; injured mammalian nerve tissue; conditions responsive to treatment through administration of a neurotrophic factor; neurological disorders; bone loss; substance related disorders; sleep/wake disorders; cardiovascular disease; and metabolic disorders such as obesity or an obesity-related disorder. Compounds of the invention modulate endocrine functions, particularly those controlled by the pituitary and hypothalamic glands, and therefore may be used to treat inovulation and infertility that may be due to insufficient release of luteinizing hormone (LH) or luteal phase defect. Compounds of the invention are also useful in the treatment of chronic heart failure.

The compounds compete with the endogenous ligands NPY and related peptides and possibly non-endogenous ligands, and bind to the NPY Y2 receptor. In addition, the compounds demonstrate antagonist activity by antagonizing the action of NPY upon binding to the Y2 receptor. Symptoms or disease states are intended to be included within the scope of “medical conditions, disorders, or diseases.” For example, “anxiety disorders” include affective disorders such as anxiety, generalized anxiety disorder (GAD), panic disorder, phobias, obsessive-compulsive disorder (OCD), stress disorders including post-traumatic stress disorder (PTSD), hemorrhagic stress, stress-induced psychotic episodes, psychosocial dwarfism, stress headaches, stress-induced immune systems disorders such as stress-induced fever, and stress-related sleep disorders, and can include eating disorders such as anorexia nervosa, bulimia nervosa, obesity, and drug addiction.

“Depression” refers to major depressive disorders, cylothymia, dysthymia, bipolar or manic disorders, and the like.

“Nerve tissue” as used herein refers to any vertebrate nerve tissue, particularly including mammalian cells of the central nervous system (CNS) and peripheral nervous system (PNS). More particularly, nerve tissue includes spinal cord neuronal structures, peripheral nervous system nerves, and even nerve cells of the brain.

“Nerve tissue injury”, “injured mammalian nerve tissue”, or “CNS or PNS nerve tissue injury” include any damage to relevant nerve tissue irrespective of cause, e.g., injuries attributable to trauma including but not limited to nerve tissue lesions, traumatically-induced compression, tumors, hemorrhage, infectious processes, spinal stenosis, or impaired blood supply.

“Treating injured mammalian nerve tissue” includes, but is not limited, to the in vivo administration of compounds, compositions, and methods of the instant invention to restore action potential or nerve impulse conduction through a nerve tissue lesion. The term may also include such administration in an effort to reduce the damaging effects of any injury to mammalian nerve tissue, whether through restoration of action potential or nerve impulse conduction, by stimulating growth or proliferation of nervous tissue, by ameliorating unwanted conditions in the extracellular microenvironment near an injury, or otherwise.

“Neurotrophic factor”, as used herein, refers to compounds that are capable of stimulating growth or proliferation of nervous tissue, including compounds of the instant invention and known neurotrophic factors described previously herein.

“Neurological disorders” include CNS disorders such as tinitus, spasticity, and neuropathic pain, supranuclear palsy, AIDS related dementias, multiinfarct dementia, neurodegenerative disorders such as Alzheimer's disease, Parkinson's disease, and Huntington's disease, head trauma, spinal cord trauma, ischemic neuronal damage, amyotrophic lateral sclerosis, and disorders of pain perception such as fibromyalgia and epilepsy.

“Bone loss” refers to enhancement of bone growth or prevention of bone loss caused by conditions such as osteoporosis, osteomalacia, Paget's disease, disorders of bone homeostasis, and the like.

“Substance related disorders” refer to misuse, addiction, or dependence disorders related to the consumption of alcohol, amphetamines (such as, for example, 3,4-methylene-dioxy-N-methylamphetamine, also known as “MDMA” or “ecstacy”), cannabis, hallucinogens (such as, for example, cocaine), inhalants, nicotine, opioids, phencydildine, narcotics, or sedatives, or combinations thereof.

“Sleep/wake disorders” include narcolepsy; sleep apnea disorders such as central sleep apnea, obstructive sleep apnea, and mixed sleep apnea; hypersomnia, including excessive daytime sleepiness (EDS), and, in particular, hypersomnia associated with narcolepsy or sleep apnea disorder; sleep/wake disturbances associated with attention deficit hyperactive disorder (ADHD); circadian rhythm abnormalities such as delayed sleep phase syndrome, advance sleep phase syndrome, non-24 hour sleep/wake disorder, jet lag, or shift-work disorder; parasomnia disorders such as somnambulism, pavor nocturnus, REM sleep behavior disorder, sleep bruxism, or sleep enuresis; sleep-related movement disorders such as sleep bruxism, restless legs syndrome, or periodic limb movement; insomnia, including extrinsic insomnia, psychophysiologic insomnia, drug-dependent insomnia, or alcohol-dependent insomnia; sleep/wake disturbances associated with mental disorders such as depression, anxiety, schizophrenia, or other psychotic disorders; sleep/wake disturbances associated with neurological disorders such as migraine, epilepsy, Parkinson's disease, or Alzheimer's disease; and sleep/wake disturbances associated with fibromyalgia, headaches, gastroesophageal reflux disease, coronary artery ischemia, cardiac arrhythmias, abnormal swallowing, choking, or laryngospasm.

“Obesity” refers to a condition in which a subject has a body mass index of greater than or equal to 30. “Over-weight” refers to a condition in which a subject has a body mass index of greater or equal to 25.0. The body mass index and other definitions are according to the “NIH Clinical Guidelines on the Identification and Evaluation, and Treatment of Overweight and Obesity in Adults” (1998).

“Obesity-related disorder” includes anorexia nervosa, wasting, AIDS-related weight loss, bulimia, cachexia, lipid disorders including hyperlipidemia and hyperuricemia, insulin resistance, noninsulin dependent diabetes mellitus (NIDDM, or Type II diabetes), insulin dependent diabetes mellitus (IDDM or Type I diabetes), diabetes-related complications including microangiopathic lesions, ocular lesions, retinopathy, neuropathy, and renal lesions, cardiovascular disease including cardiac insufficiency, coronary insufficiency, and high blood pressure, atherosclerosis, atheromatous disease, stroke, hypertension, Syndrome X, gallbladder disease, osteoarthritis, sleep apnea, forms of cancer such as uterine, breast, colorectal, kidney, and gallbladder, high cholesterol levels, complications of pregnancy, menstrual irregularities, hirsutism, muscular dystrophy, infertility, and increased surgical risk.

“Cardiovascular disease” includes, for example, cardiac arrhythmia, post-myocardial infarction, and heart failure.

Thus, the pharmaceutical agents may be used to treat subjects diagnosed with or suffering from a disease, disorder, or condition mediated through NPY Y2 activity. The term “treat” or “treating1’ as used herein is intended to refer to administration of at least one agent of the invention or a composition of the invention to a subject for the purpose of effecting a therapeutic or prophylactic benefit through modulation of NPY Y2 activity.

Treating includes reversing, ameliorating, alleviating, inhibiting the progress of, lessening the severity of, or preventing a disease, disorder or condition, or one or more symptoms of such disease, disorder or condition mediated through modulation of NPY Y2 activity. The term “subject” refers to a mammalian patient in need of such treatment, such as a human. “Modulators” include both inhibitors and activators, where “inhibitors” refer to compounds that decrease, prevent, inactivate, desensitize or down-regulate NPY Y2 expression, activity or function, and “activators” are compounds that increase, activate, facilitate, sensitize, or up-regulate NPY Y2 expression, activity, or function.

Accordingly, the invention relates to methods of using the pharmaceutical agents described herein to treat subjects diagnosed with or suffering from a disease, disorder, or condition mediated through NPY Y2 activity, such as: anxiety disorders and depression; injured mammalian nerve tissue: conditions responsive to treatment through administration of a neurotrophic factor; neurological disorders; bone loss; substance related disorders; metabolic disorders such as obesity or an obesity-related disorder; inovulation and infertility that may be due to insufficient release of luteinizing hormone (LH) or luteal phase defect; and cardiovascular disease, cardiac arrhythmia, post-myocardial infarction, or chronic heart failure. In particular, the invention relates to methods of using the pharmaceutical agents described herein to treat subjects diagnosed with or suffering from a disease, disorder, or condition mediated through NPY Y2 activity, such as anxiety and alcoholism.

In certain preferred embodiments of the method, the disease, disorder, or medical condition is selected from: anxiety disorders and depression; a condition requiring treatment of injured mammalian nerve tissue; a condition amenable to treatment through administration of a neurotrophic factor; a neurological disorder; bone loss; substance related disorders; sleep/wake disorders; cardiovascular disease such as cardiac arrhythmia, post-myocardial infarction, or heart failure; obesity; an obesity-related disorder; and a condition related to an endocrine function including inovulation and infertility.

Furthermore, the agents of the invention may be useful in the prevention, treatment or delay of progression of disorders of the gastro-intestinal tract mediated full or in part by NPY Y2 receptors.

Disorders of the gastro-intestinal tract include Gastro-Esophageal Reflux Disease (GERD), idiopathic and diabetic gastroparesis, post-operative ileus, and Functional Gastro-intestinal Disorders (FGIDs).

GERD is defined as chronic symptoms or mucosal damage produced by the abnormal reflux in the esophagus. This is commonly due to transient or permanent changes in the barrier between the esophagus and the stomach. Gastroparesis, also called delayed gastric emptying, is a medical condition consisting of a paresis (partial paralysis) of the stomach (“gastro-”), resulting in food remaining in the stomach for a longer period of time than normal, and is often associated with feelings of discomfort. Post-operative Ileus is defined as failure of aboral passage of intestinal contents due to transient impairment of GI motility following abdominal surgery.

FGIDs are defined as chronic or recurrent conditions associated with abdominal symptoms without organic cause using conventional diagnostic measures. A cardinal symptom present in many FGIDs is visceral pain and/or discomfort. FGIDs include functional dyspepsia (FD), functional heartburn (a subset of GERD), irritable bowel syndrome (IBS) associated with constipation and/or diarrhea, functional bloating, functional diarrhea, chronic constipation, functional disturbancies of the biliary tract as well as other conditions according to Gut 1999; Vol. 45 Suppl. II.

The agents of the present invention may be useful for the prevention of the above-mentioned conditions and disorders.

The agents of the present invention may be useful for the treatment of the above-mentioned conditions and disorders.

The agents of the present invention may be useful for the delay of progression of the above-mentioned conditions and disorders.

The usefulness of the agents of the invention in the treatment of the above-mentioned disorders can be confirmed in a range of standard tests including those indicated below:

Activity of the agents of the invention in GERD can be demonstrated in standard models that measure gastric distension-induced transient lower esophageal sphincter relaxations (TLESRs) in dogs according to Stakeberg, J. and Lehmann, A. Neurogastroenterol. Mot. (1999) 11: 125-132. At doses of about 0.03 to about 10 mg/kg i.p., s.c. or p.o., selected agents of the invention may reduce the occurrence of TLESRs.

Activity of the agents of the invention in gastroparesis can be demonstrated in standard models that measure gastric emptying such as the breath test (methodology according to Schoonjans R. et al., Neurogastroenterol. Mot. (2002) 14: 287-293) or near infrared fluorescent imaging (methodology according to Gremlich et al., J. Mol. Imaging (2004) 3: 303-311). At doses of about 0.03 to about 10 mg/kg i.p., s.c. or p.o., selected agents of the invention may increase gastric emptying in either mice, rats or dogs.

Activity of the agents of the invention in functional dyspepsia can be demonstrated by a model that assesses fasted gastric tone and gastric accommodation to a meal in rats by measuring the intragastric pressure during meal infusion (methodology according to Janssen P. et al., Scand J. Gastroenterology (2007) 43: 34-43). At doses of about 0.03 to about 10 mg/kg i.p., s.c. or p.o., selected agents of the invention may decrease gastric pressure during meal infusion.

Furthermore, the activity of the agents of the invention in functional dyspepsia can be demonstrated in a model of fasted gastric tone and gastric accommodation to meal in dogs (methodology according to Lei et al., Dig. Dis. Sci. (2005) 50:2134-40). At doses of about 0.03 to about 10 mg/kg p.o., selected agents of the invention may increase the gastric volume in fasting conditions indicative of a reduced gastric tone.

Activity of the agents of the invention in post-operative ileus can be demonstrated in standard models to measure gastrointestinal motility after abdominal surgery (according to Huge, A. et al., J. Surg. Res (1998) 74: 112-118). At doses of about 0.03 to about 10 mg/kg i.p., s.c. or p.o., selected agents of the invention may induce a faster restauration of gastrointestinal motility as compared to vehicle/placebo treatment.

For the above-mentioned indications, the appropriate dosage will vary depending on, e. g., the compound employed, the host, the mode of administration and the nature and severity of the condition, disorder or disease. However, in general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range of from about 10 to about 2000, preferably from about 10 to about 200, mg of an agent of the invention conveniently administered, for example, in divided doses up to four times a day or in sustained release form.

An agent of the invention may be administered by any conventional route, in particular enterally, preferably orally, for example in the form of tablets or capsules, or parenterally, for example in the form of injectable solutions or suspensions.

In accordance with the foregoing, in a further aspect, the invention relates to an agent of the invention, for use as a medicament, e. g. for the treatment or prevention of conditions, disorders or diseases that can be modulated or are mediated by NPY Y2 receptors.

In a further aspect, the invention relates to the use of an agent of the invention as active ingredient in a medicament, e. g. for the treatment or prevention of conditions, disorders or diseases, that can be modulated or are mediated by NPY Y2 receptors.

In a further aspect, the invention relates to a pharmaceutical composition comprising an agent of the invention as active ingredient in association with at least one pharmaceutical carrier or diluent. Such compositions may be manufactured in conventional manner. Unit dosage forms contain, for example, from about 1 to about 1000, preferably from about 1 to about 500, mg of an agent of the invention.

In a further aspect, the invention relates to the use of an agent of the invention for the manufacture of a medicament for the treatment or prevention of conditions, disorders or diseases that can be modulated or are mediated by NPY Y2 receptors.

In a further aspect, the invention relates to a method for the treatment or prevention of conditions, disorders or diseases that can be modulated or are mediated by NPY Y2 receptors, in a subject in need of such treatment, which comprises administering to such subject a therapeutically effective amount of an agent of the invention.

In a further aspect, the invention relates to pharmaceutical compositions each comprising: (a) an effective amount of an agent selected from compounds of Formula I or I′ and pharmaceutically acceptable salts, pharmaceutically acceptable prodrugs, and pharmaceutically active metabolites thereof; and (b) a pharmaceutically acceptable excipient.

In a treatment method according to the invention, an effective amount of at least one pharmaceutical agent according to the invention is administered to a subject suffering from or diagnosed as having such a disease, disorder, or condition. An “effective amount” means an amount or dose sufficient to generally bring about the desired therapeutic or prophylactic benefit in patients in need of such treatment. Effective amounts or doses of the agents of the present invention may be ascertained by routine methods such as modeling, dose escalation studies, or clinical trials, and by taking into consideration routine factors, e.g., the mode or route of administration or drug delivery, the pharmacokinetics of the agent, the severity and course of the disease, disorder, or condition, the subject's previous or ongoing therapy, the subjects health status and response to drugs, and the judgment of the treating physician. An exemplary dose is in the range of from about 0.001 to about 200 mg of agent per kg of subject's body weight per day, preferably about 0.05 to 100 mg/kg/day, or about to 35 mg/kg/day, in single or divided dosage units (e.g., BID, TID, OID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment.

For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

The agents of the invention can be administered alone or as combination with other pharmaceutical agents effective, e. g., in the treatment or prevention of conditions, disorders or diseases mentioned above. Such pharmaceutical combinations may be in the form of a unit dosage form, whereby each unit dosage will comprise a predetermined amount of the two components in admixture with at least one pharmaceutical carrier or diluent. Alternatively, the combination may be in the form of a package containing the two components separately, e. g. a pack or dispenser-device adapted for the concomitant or separate administration of the two active agents, wherein these agents are separately arranged. In a further aspect, the invention relates to such pharmaceutical combinations. The additional compounds may be co-administered separately with an agent of Formula I or I′ or included with such an agent as an additional active ingredient in a pharmaceutical composition according to the invention. In an exemplary embodiment, additional active compounds are those that are known or discovered to be effective in the treatment of conditions, disorders, or diseases mediated by NPY Y2 activity, such as another NPY Y2 modulator or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the agent according to the invention. In one illustrative embodiment, a composition according to the invention may contain one or more additional active ingredients selected from anxiolytics, antidepressants, and hypnotics.

The agents of the invention are used, alone or in combination with one or more other active ingredients, to formulate pharmaceutical compositions of the invention. A pharmaceutical composition of the invention comprises: (a) an effective amount of at least one pharmaceutical agent in accordance with the invention; and (b) a pharmaceutically acceptable excipient.

A “pharmaceutically acceptable excipient” refers to a substance, that is not toxic, biologically intolerable, or otherwise biologically unsuitable for administration to a subject, such as an inert substance, added to a pharmacological composition or otherwise used as a vehicle, carrier, or diluent, to facilitate administration of a pharmaceutical agent and that is compatible therewith Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the pharmaceutical agents may be prepared using suitable pharmaceutical excipients and compounding techniques now or later known or available to those skilled in the art. The compositions may be administered in the inventive methods by oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension.

To prepare the oral compositions, the agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the active ingredient mixed with pharmaceutically acceptable excipients such as inert diluents, disintegrating agents, binding agents, lubricating agents, sweetening agents, flavoring agents, coloring agents, and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose. and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating. Capsules for oral administration include hard and soft gelatin capsules.

To prepare hard gelatin capsules, active ingredient may be mixed with a solid, semi-solid, or liquid diluent. Soft gelatin capsules may be prepared by mixing the active ingredient with water, an oil subh as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The agents of this invention may also be administered by non-oral routes. For example, the compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil.

Suitable aqueous vehicles include Ringer's solution and isotonic sodium chloride. Such forms will be presented in unit-dose form such as ampulesor disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of agent, admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdenmal delivery. Agents may alternatively be administered in methods of this invention by inhalation, via the nasal or oral routes, e.g., in a spray formulation also containing a suitable carrier.

Exemplary agents useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula I or I′.

Assay Methods:

Preparation of Membranes: CHO-C4 cells expressing recombinant human NPY Y2 receptors were used to prepare membranes for the GTPγS assay. Cells were grown to 80-95% confluency on 15-cm (225 cm²) tissue culture plates. After aspiration of the culture medium, cells were washed twice with 18 ml ice-cold phosphate-buffered saline (PBS), scraped off and suspended in 3 ml ice-cold PBS in pre-cooled centrifuge tubes. The dishes were rinsed with 2 ml ice-cold PBS per dish and the washings were combined with the PBS cell suspension from above. Cells pooled from 5-7 dishes were centrifuged for 5 min at 10,000 rpm (12,000 g) in a Sorvall RC5B centrifuge using an SS34 rotor at 4° C. The cell pellet was resuspended in 5 ml ice-cold buffer (20 mM HEPES, 10 mM EDTA; pH 7.4) by vortexing (2-5 sec), homogenized using a Polytron (step 4 for 20-30 sec), and ice-cold buffer added to 25 ml. The suspension was centrifuged again for 20 min at 18,000 rpm (39,000 g) at 4° C. and the pellet resuspended in 5 ml ice-cold buffer (20 mM HEPES, 0.1 mM EDTA; pH 7.4) by vortexing (2-5 sec), homogenized with a Polytron (step 4 for 10 sec), and ice-cold buffer added to 25 ml. The suspension was centrifuged a third time for 20 min at 18,000 rpm (39,000 g) at 4° C. The pellet was resuspend in 1 ml ice-cold buffer (20 mM HEPES, 0.1 mM EDTA; pH 7.4) by vortexing (5-8 sec). Two to five resuspended pellets were combined and homogenized using a Polytron (step 4 for 15-25 sec). A small aliquot (20-50 μl) was removed for protein determination by the Coomassie Plus Protein Assay Reagent (Pierce) using BSA as standard. The membrane suspension was aliquoted in pre-cooled (on dry ice) Eppendorf tubes (0.25-1 ml/tube affording approximately 0.5-2 mg of membrane protein/tube). The pellets were frozen and stored at −80° C.

Scintillation proximity [³⁵S]GTPγS binding assay: Frozen membranes from CHO-C4 cells expressing recombinant human NPY Y2 receptors (2 mg for four 96-well plates) were thawed on ice. Thawed membranes were pipetted into 10 ml of assay buffer (20 mM HEPES, 10 mM MgCl2, 100 mM NaCl, pH 7.4) and homogenized briefly using a Polytron. The final assay mixture was prepared in 96-well microtiter plates (Isoplate Wallac, Perkin Elmer). The composition of the assay mixtures in a final volume of 250 μl per well was as follows: 20 mM HEPES, 10 mM MgCl2, 100 mM NaCl, pH 7.4, 30 μM GDP, 1 mg/ml BSA (added fresh), 5 μg membrane protein, 1.5 mg Wheatgerm agglutinin SPA beads (Amersham), 0.45 nM [³⁵S]GTPγS (Amersham, SJ1308. 1000 Ci/mmol, stabilized solution), and the test compounds (agonists and/or antagonists) at the appropriate concentrations. The samples were incubated at room temperature for 90 min by shaking, after which the SPA beads were sedimented by centrifugation in an Eppendorf 5804 centrifuge at 2700 rpm for 10 min at room temperature. After 60 min the plates were counted in a TopCount (Canberra). Basal [³⁵S]GTPγS binding was measured in the absence of agonist (NPY). Non-specific binding was measured in the presence of excess (10 μM) unlabelled GTPγS (Sigma). Nonspecific binding never exceeded 10% of basal binding and was thus not subtracted from the experimental data. Antagonists were tested for the inhibition of 0.5 nM NPY-stimulated [³⁵S]GTPγS binding. Antagonist inhibition curves were analyzed by non-linear regression using GraphPad Prism software (Version 4.0, GraphPad Software Inc., CA, USA).

In order to illustrate the invention, the following examples are included. These examples do not limit the invention. They are only meant to suggest a method of practicing the invention. Those skilled in the art may find other methods of practicing the invention, which are obvious to them. However, those methods are deemed to be within the scope of this invention. Unless otherwise noted, the materials used in the examples were obtained from readily available commercial sources or synthesized by standard methods known to those skilled in the art. A list of Abbreviations used is given below:

Boc tert-butyloxycarbonyl

DCC Dicyclohexylcarbodiimide

DCE dichloroethane

DCM dichloromethane

DMF N,N′dimethylformamide

LC liquid chromatography

r.t. retention time (LC/MS)

RT room temperature

TFA Trifluoroacetic acid

THF tetrahydrofuran

HPLC specificity: Gilson 331 pumps coupled to a Gilson UV/VIS 152 detector and a Finnigan AQA spectrometer (ESI), a 50 μL loop injection valve and a Waters XTerra MS C18 3.5 μm 4.6×50 mm column running a gradient Water+0.05% TFA/Acetonitrile+0.05% TFA from 95/5 to 10/90 over 8 min with a flux of 1.5 mL/min

### EXAMPLE 1

### 1-[4-(4-Benzhydryl-piperazin-1-yl)-3-cyano-phenyl]-3-(3,5-dimethyl-isoxazol-4-yl)-urea

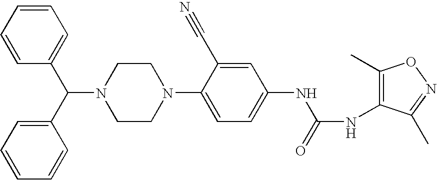

### Step a: 4-(2-Cyano-4-nitro-phenyl)-piperazine-1-carboxylic acid tert-butyl ester

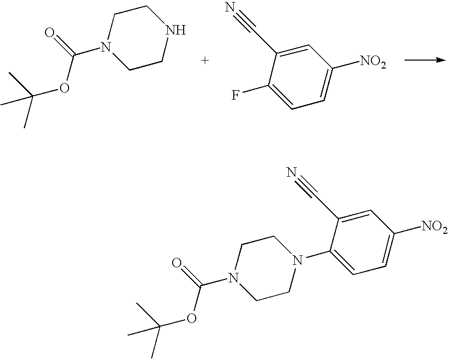

To a mixture of 1-Boc-piperazine (5.61 g, 30.1 mmol) and potassium carbonate (4.2 g, 1 eq) in DMF (50 mL), 2-fluoro-5-nitro-benzonitrile (5.0 g, 1.0 eq) was added. The resulting mixture was stirred at 50° C. for 2 hours. The mixture was poured into water and the resulting precipitate was filtered off and washed with water. After high-vacuum drying, 4-(2-cyano-4-nitro-phenyl)-piperazine-1-carboxylic acid tert-butyl ester (8.66 g, 86.6% yield) was obtained as a yellow solid which will be used without further purification.

LC/MS: r.t. 6.22 min, 274.2 [M+H+CH₃CN-Boc]

### Step b: 5-Nitro-2-piperazin-1-yl-benzonitrile TFA salt

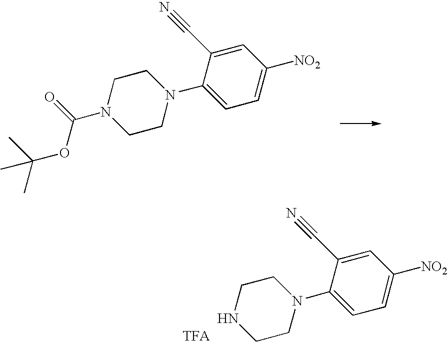

4-(2-Cyano-4-nitro-phenyl)-piperazine-1-carboxylic acid tert-butyl ester (8.5 g, 25.6 mmol) was dissolved in DCM/TFA (10/1, 55 mL) and the resulting solution was stirred at RT for 3 hours. The mixture was concentrated to around 10 mL in vacuo and then 100 mL of Et₂O was added. The resulting precipitate was filtered off and washed with diethyl ether. After high-vacuum drying, 5-nitro-2-piperazin-1-yl-benzonitrile TFA salt (8.2 g, 97.1% yield) was obtained as a beige solid which will be used without further purification.

LC/MS: r.t. 3.06 min, 274.2 [M+H+CH₃CN]

### Step c: 2-(4-Benzhydryl-piperazin-1-yl)-5-nitro-benzonitrile

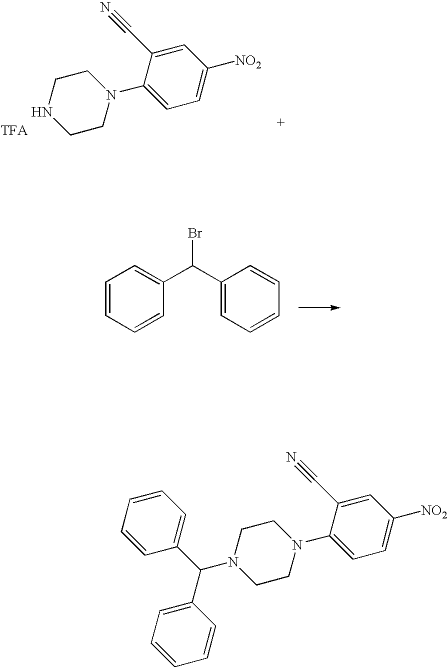

A mixture of 5-nitro-2-piperazin-1-yl-benzonitrile TFA salt (800 mg, 2.42 mmol), bromodiphenylmethane (672 mg, 1.1 eq) and potassium carbonate (744 mg, 2.2 eq) in DMF (20 mL) was stirred at RT for 16 hours. The mixture was poured into water and the resulting solid was filtered off and washed with water before to be solubilised in DCM. The organic phase was dried over sodium sulfate and then concentrated in vacuo to afford a crude brown-orange powder. The crude powder was sonicated in pentane and then the resulting precipitate was filetered off and washed with pentane. After high-vacuum drying, 2-(4-benzhydryl-piperazin-1-yl)-5-nitro-benzonitrile (700 mg, 72.5% yield) was obtained as an orange-brown solid which will be used without further purification.

LC/MS: r.t. 4.84 min, 399.2 [M+H]

### Step d: 5-Amino-2-(4-benzhydryl-piperazin-1-yl)-benzonitrile

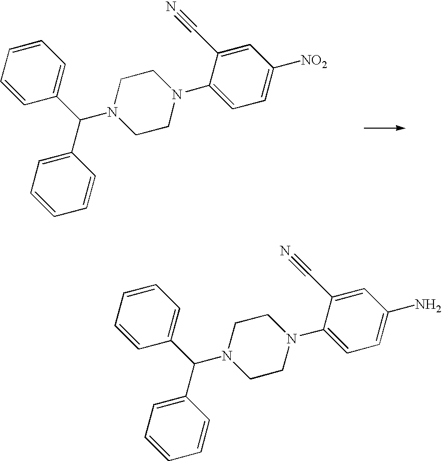

A mixture of 2-(4-benzhydryl-piperazin-1-yl)-5-nitro-benzonitrile (700 mg, 1.76 mmol) and tin chloride hydrate (2.38 g, 6 eq) in EtOH/AcOEt (1/1, 20 mL) was refluxed for 4 hours. The mixture was poured into water and the pH was adjusted to pH 9 by addition of a saturated solution of NaHCO₃. The aqueous phase was extracted with AcOEt. The organic phase were combined, dried over sodium sulfate and concentrated in vacuo to afford a crude red oil. This oil was sonicated in pentane/diethyl ether (1/1) and the resulting precipitate was filetered off and washed with pentane/diethyl ether (1/1). After high-vacuum drying, 5-amino-2-(4-benzhydryl-piperazin-1-yl)-benzonitrile (560 mg, 86.5% yield) was obtained as a beige-pink solid which will be used without further purification.

LC/MS: r.t. 4.02 min, 369.2 [M+H]

### Step e: 1-[4-(4-Benzhydryl-piperazin-1-yl)-3-cyano-phenyl]-3-(3,5-dimethyl-isoxazol-4-yl)-urea

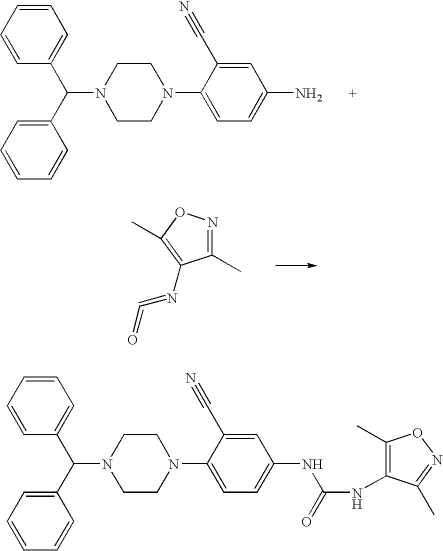

A mixture of 5-amino-2-(4-benzhydryl-piperazin-1-yl)-benzonitrile (150 mg, 0.40 mmol) and 3,5-dimethyl isoxazol-4-yl isocyanate (55 mg, 1 eq) in DCE (10 mL) was stirred at RT for 2 hours. To the mixture NaOH 2N was added and the organic phase was separated. The aqueous phase was extracted with DCM. The organic phases were combined, dried over sodium sulfate and concentrated in vacuo to afford a brown powder. The crude product was purified by flash chromatography on silica gel using DCM/MeOH+10% NH₄OH (100 to 90/10) as solvent system. From the purification, 1-[4-(4-benzhydryl-piperazin-1-yl)-3-cyano-phenyl]-3-(3,5-dimethyl-isoxazol-4-yl)-urea (89 mg, 43% yield) was isolated as a white powder.

LC/MS: r.t. 4.38 min, 507.5 [M+H]

### EXAMPLE 2

### Tetrahydro-furan-3-carboxylic acid [4-(4-benzhydryl-piperazin-1-yl)-3-cyano-phenyl]-amide

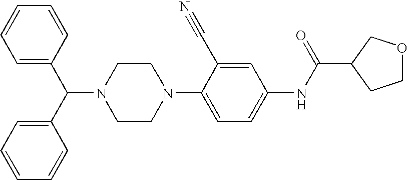

To a mixture of Example 1, step d (150 mg, 0.40 mmol) and DCC (277 mg, 3.3 eq) in DCE (10 mL) was stirred at RT for 1 hour. Then, tetrahydro-3-furoic acid (156 mg, 3.3 eq) was added and the resulting mixture was stirred at RT for 16 hours. To the mixture NaOH 2N was added and the organic phase was separated. The aqueous phase was extracted with DCM. The organic phases were combined, dried over sodium sulfate and concentrated in vacuo to afford a beige brown powder. The crude powder was sonicated in AcOEt/diethyl ether (1/1) and the resulting precipitate was filtered off and washed with diethyl ether. After high-vacuum drying, tetrahydro-furan-3carboxylic acid [4-(4-benzhydryl-piperazin-1-yl)-3cyano-phenyl]-amide (96 mg, 50.5% yield) was obtained as a white powder.

LC/MS: r.t. 4.17 min, 467.5 [M+H]

### EXAMPLE 3

### N-[4-(4-Benzhydryl-piperazin-1-yl)-3-cyano-phenyl]-2-ethyl-butyramide

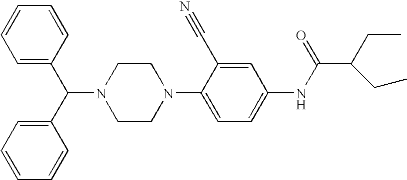

To a mixture of Example 1, step d (150 mg, 0.40 mmol) and DCC (277 mg, 3.3 eq) in DCE (10 mL) was stirred at RT for 1 hour. Then, 2-ethylbutyric acid (156 mg, 3.3 eq) was added and the resulting mixture was stirred at RT for 16 hours. To the mixture NaOH 2N was added and the organic phase was separated. The aqueous phase was extracted with DCM. The organic phases were combined, dried over sodium sulfate and concentrated in vacuo to afford a pink powder. The crude product was purified by flash chromatography on silica gel using DCM/MeOH (100 to 95/5) as solvent system. From the purification, N-[4-(4-Benzhydryl-piperazin-1-yl)-3-cyano-phenyl]-2-ethyl-butyramide (36 mg, 19% yield) was isolated as a white powder.

LC/MS: r.t. 4.61 min, 467.4 [M+H]

### EXAMPLE 4

### 1-[4-(4-Benzhydryl-piperazin-1-yl)-3-fluoro-phenyl]-3-(3,5-dimethyl-isoxazol-4-yl)-urea

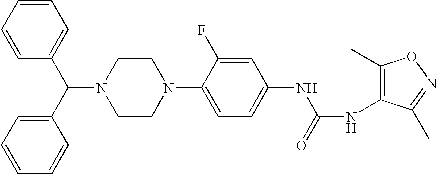

Starting material 4-(4-benzhydryl-piperazin-1-yl)-3-fluoro-phenylamine was prepared accordingly to Example 1 step a to d with replacing 2-fluoro-5nitro-benzonitrile by 3,4-difluoro-nitrobenzene.

Synthesis analogous to Example 1 step e with starting with 4-(4-benzhydryl-piperazin-1-yl)-3-fluoro-phenylamine (150 mg, 0.415 mmol) and 3,5-dimethyl isoxazol-4-yl isocyanate. 1-[4-(4-Benzhydryl-piperazin-1-yl)-3-fluorophenyl]-3-(3,5-dimethyl-isoxazol-4-yl)-urea (163 mg, 78.6% yield) was obtained as a white powder.

LC/MS: r.t. 4.28 min, 499.6 [M+H]

### EXAMPLE 5

### Tetrahydro-furan-3-carboxylic acid [4-4-benzhydryl-piperazin-1-yl)-3-cyano-phenyl]-amide

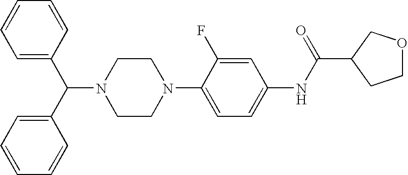

Synthesis analogous to Example 2 with starting with 4-(4-benzhydryl-piperazin-1-yl)-3-fluoro-phenylamine (150 mg, 0.415 mmol) and tetrahydro-3-furoic acid. Tetrahydrofuran-3-carboxylic acid [4-(4-benzhydryl-piperazin-1-yl)-3-cyano-phenyl]-amide (117 mg, 61.4% yield) was obtained as a white powder.

LC/MS: r.t. 4.17 min, 460.4 [M+H]

### EXAMPLE 6

### N-[4-4-Benzhydryl-piperazin-1-yl)-3-fluoro-phenyl]-2-ethyl-butyramide

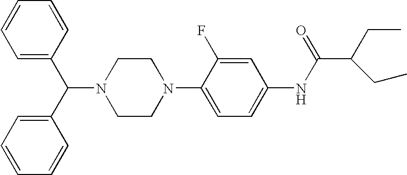

Synthesis analogous to Example 3 with starting with 4-(4-benzhydryl-piperazin-1-yl)-3-fluoro-phenylamine (150 mg, 0.415 mmol) and 2-ethylbutyric acid. N-[4-(4-Benzhydryl-piperazin-1-yl)-3-fluoro-phenyl]-2-ethyl-butyramide (75 mg, 39.3% yield) was obtained as a white powder.

LC/MS: r.t. 4.74 min, 460.4 [M+H]

### EXAMPLE 7

### 1-(4-{4-[Bis-4-fluoro-phenyl)-methyl]-piperazin-1-yl}-3-cyano-phenyl)-3-(3,5-dimethyl-isoxazol-4-yl)-urea

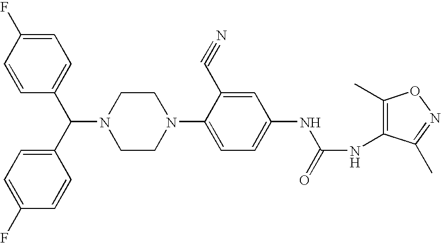

### Step a: 2-{4-[Bis-(4-fluoro-phenyl)-methyl]-piperazin-1-yl}-5-nitro-benzonitrile

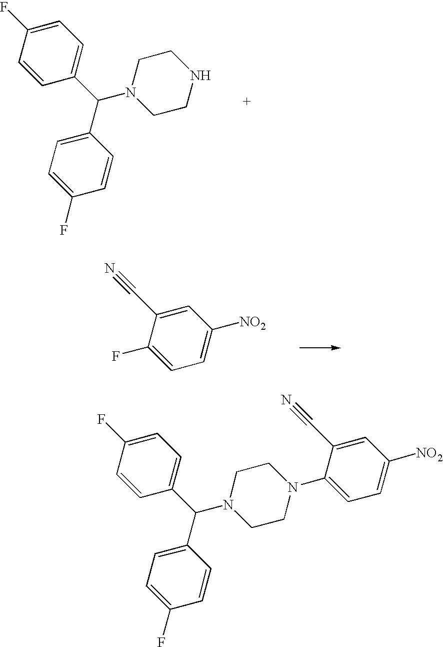

A mixture of 1-[bis(4-fluorophenyl)methyl]piperazine (5.0 g, 17.3 mmol), 2-fluoro-5-nitro-benzonitrile (2.88 g, 1.0 eq) and potassium carbonate (2.64 g, 1.1 eq) in DMF (50 mL) was stirred at 50° C. for 2 hours. The mixture was poured into water and the precipitate was filtered off and washed with water before to be solubilised in DCM. The organic phase was dried over sodium sulfate and concentrated in vacuo to afford a reddish gum. This gum was sonicated in DCM/Hexanes (2/8) and then the precipitate was filtered off and washed with Hexanes. After high-vacuum drying, 2-{4-[bis-(4-fluoro-phenyl)-methyl]-piperazin-1-yl}-5-nitro-benzonitrile (5.8 g, 77% yield) was obtained as an ocre solid which will be used without further purification.

LC/MS: r.t. 5.33 min, 435.3 [M+H]

### Step b: 5-Amino-2-{4-[bis-(4-fluoro-phenyl)-methyl]-piperazin-1-yl}-benzonitrile

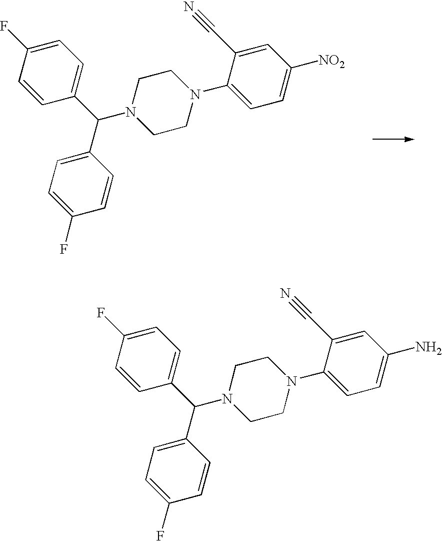

Synthesis analogous to Example 1 step d. 5-Amino-2-{4-[bis-(4-fluoro-phenyl)-methyl]-piperazin-1-yl}-benzonitrile (4.5 g, 83.3% yield) was obtained as a light pink powder.

LC/MS: r.t. 4.38 min, 405.3 [M+H]

### Step c: 1-(4-{4-[Bis-(4-fluoro-phenyl)-methyl]-piperazin-1-yl}-3-cyano-phenyl)-3-(3,5-dimethyl-isoxazol-4-yl)-urea

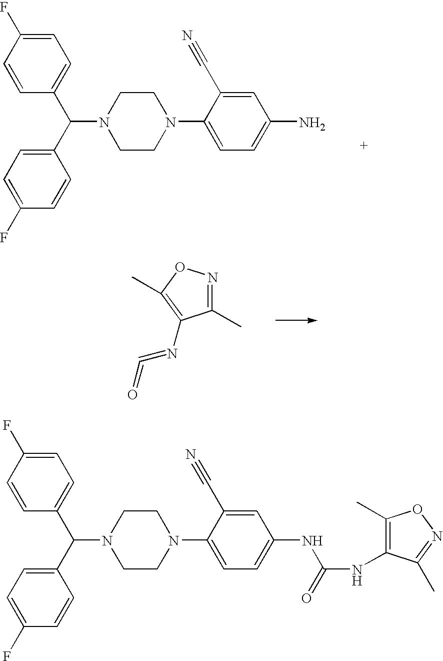

Synthesis analogous to Example 1 step e with starting with 5-amino-2-{4-[bis-(4-fluoro-phenyl)-methyl]-piperazin-1-yl}-benzonitrile (250 mg, 0.62 mmol) and 3,5-dimethyl isoxazol-4-yl isocyanate. 1-(4-{4-[Bis-(4-fluoro-phenyl)-methyl]-piperazin-1-yl}-3-cyano-phenyl)-3-(3,5-dimethyl-isoxazol-4-yl)-urea (220 mg, 65.6% yield) was obtained as a light pink powder.

LC/MS: r.t. 5.03 min, 543.5 [M+H]

### EXAMPLE 8

### 1-(4-{4-[Bis-(4-fluoro-phenyl)-methyl]-piperazin-1-yl}-3-fluoro-phenyl)-3-(3,5-dimethyl-isoxazol-4-yl)-urea

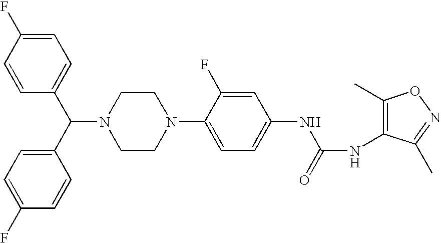

Starting material 4-{4-[bis-(4-fluoro-phenyl)-methyl]-piperazin-1-yl}-3-fluoro-phenyl-amine was prepared accordingly to Example 7 step a to b with replacing 2-fluoro-5-nitro-benzonitrile by 3,4-difluoro-nitrobenzene.

Synthesis analogous to Example 1 step e with starting with 4-{4-[bis-(4-fluoro-phenyl)-methyl]-piperazin-1-yl}-3-fluoro-phenylamine (250 mg, 0.63 mmol) and 3,5-dimethyl isoxazol-4-yl isocyanate.

The title compound (225 mg, 66.8% yield) was obtained as an off-white powder.

LC/MS: r.t. 5.00 min, 536.6 [M+H]

### EXAMPLE 9

### 3,5-Dimethyl-isoxazole-4-carboxylic acid [4-(4-benzhydryl-piperazin-1-yl)-3-fluoro-phenyl]-amide

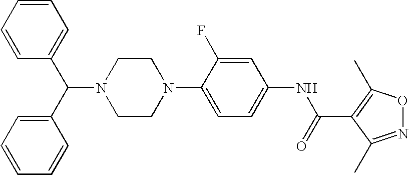

To a mixture of 4-(4-benzhydryl-piperazin-1-yl)-3-fluoro-phenylamine (140 mg, 0.39 mmol) and triethylamine (0.103 mL, 1.96 eq) in DCE (10 mL) was added 3,5-dimethyl-isoxazole-4-carbonyl chloride (75 mg, 1.2 eq, prepared from reaction of 3,5-dimethyl-isoxazole-4-carboxylic acid with oxalyl chloride in DCM under DMF catalysis). The resulting mixture was stirred at RT for 2 hours. NaOH 2N was added and the aqueous phase was extracted with DCM. The organic phases were combined, dried over sodium sulfate and concentrated in vacuo to afford a crude beige powder. The crude material was purified by flash chromatography on silica gel using Hexanes/DCM (100/0 to 50/50) as solvent system. From the purification, 3,5-dimethyl-isoxazole-4-carboxylic acid [4-(4-benzhydryl-piperazin-1-yl)-3-fluoro-phenyl]-amide (66 mg, 35% yield) was isolated as a white powder.

LC/MS: r.t. 4.80 min, 485.3 [M+H]

### EXAMPLE 10

### N-[4-(4-Benzhydryl-piperazin-1-yl)-3-fluoro-phenyl]-2-methyl-nicotinamide

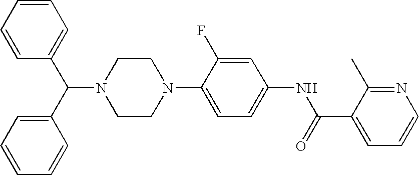

Synthesis analogous to Example 9 with starting with 4-(4-benzhydryl-piperazin-1-yl)-3-fluoro-phenylamine (150 mg, 0.41 mmol) and 2-methyl-nicotinic acid. N-[4-(4-Benzhydryl-piperazin-1-yl)-3-fluoro-phenyl]-2-methyl-nicotinamide (55 mg, 27.6% yield) was obtained as a white powder.

LC/MS: r.t. 4.07 min, 481.1 [M+H]

### EXAMPLE 11

### 1-Methyl-1H-pyrrole-2-carboxylic acid [4-(4-benzhydryl-piperazin-1-yl)-3-fluoro-phenyl]-amide

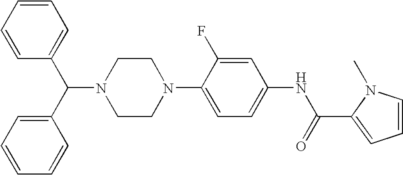

Synthesis analogous to Example 9 with starting with 4-(4-benzhydryl-piperazin-1-yl)-3-fluoro-phenylamine (200 mg, 0.55 mmol) and 1-methylpyrrole-2-carboxylic acid. 1-Methyl-1H-pyrrole-2-carboxylic acid [4-(4-benzhydryl-piperazin-1-yl)-3-fluoro-phenyl]-amide (181 mg, 69.8% yield) was obtained as a beige powder.

LC/MS: r.t. 4.96 min, 469.3 [M+H]

### EXAMPLE 12

### 2-Methyl-2H-pyrazole-3-carboxylic acid [4-(4-benzhydryl-piperazin-1-yl)-3-fluoro-phenyl]-amide

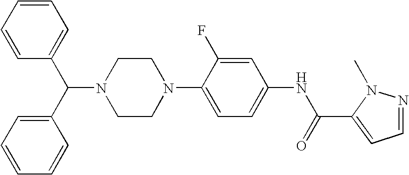

Synthesis analogous to Example 9 with starting with 4-(4-benzhydryl-piperazin-1-yl)-3-fluoro-phenylamine (200 mg, 0.55 mmol) and 1-methyl-1H-pyrazole-5-carboxylic acid. 2-Methyl-2H-pyrazole-3-carboxylic acid [4-4-benzhydryl-piperazin-1-yl)-3-fluoro-phenyl]-amide (178 mg, 68.5% yield) was obtained as a light pink powder.

LC/MS: r.t. 4.69 min, 470.3 [M+H]

### EXAMPLE 13

### 1-[4-(4-Benzhydryl-piperidin-1-yl)-3-cyano-phenyl]-3-3,5-dimethyl-isoxazol-4-yl)-urea

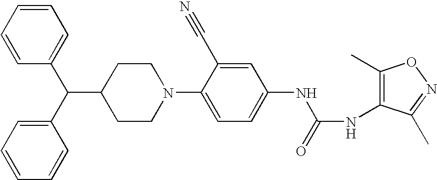

### Step a: 2-(4-Benzhydryl-piperidin-1-yl)-5-nitro-benzonitrile

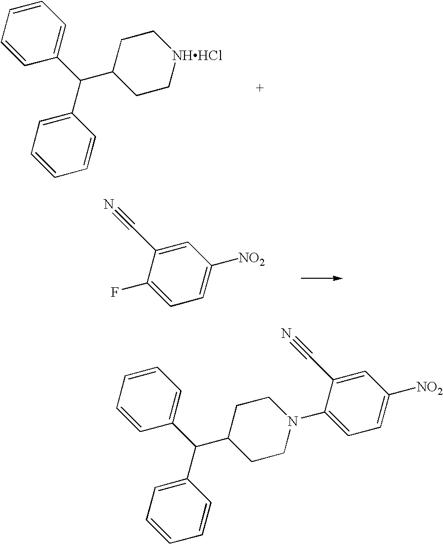

A mixture of 4-benzhydryl-piperidine hydrochloride salt (2.2 g, 7.64 mmol), 2-fluoro-5-nitro-benzonitrile (1.77 g, 1.4 eq) and potassium carbonate (2.72 g, 2.55 eq) in DMF (25 mL) was stirred at 50° C. for 2 hours. The mixture was quenched with water and the pH was adjusted to pH 9-10 by addition of NaOH 2N. The aqueous phase was extracted with ethyl acetate. The organic phases were combined, washed with brine, dried over sodium sulfate and concentrated in vacuo to afford a crude orange oil (5.1 g). The crude material was purified by flash chromatography on silica gel using Hexanes/AcOEt (100/0 to 50/50) as solvent system. From the purification, 2-(4-benzhydryl-piperidin-1-yl)-5-nitro-benzonitrile (2.54 g, 83.6% yield) was isolated as an orange powder.

LC/MS: r.t 5.78 min, 398.3 [M+H] gradient from 50% to 100% B

### Step b: 5-Amino-2-(4-benzhydryl-piperidin-1-yl)-benzonitrile

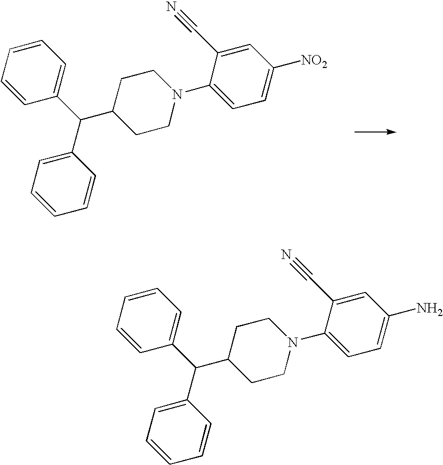

Synthesis analogous to Example 1 step d with starting with 2-(4-benzhydryl-piperidin-1-yl)-5-nitro-benzonitrile (2.5 g, 6.28 mmol). 5-Amino-2-(4-benzhydryl-piperidin-1-yl)-benzonitrile (2.2 g, 95% yield) was obtained as a beige-brown powder.

LC/MS: r.t. 6.10 min, 368.2 [M+H]

### Step c: 1-[4-(4-Benzhydryl-piperidin-1-yl)-3-cyano-phenyl]-3-(3,5-dimethyl-isoxazol-4-yl)-urea

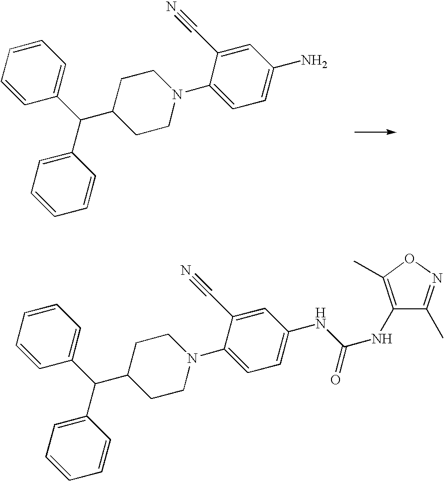

Synthesis analogous to Example 1 step e with starting with 5-amino-2-(4-benzhydryl-piperidin-1-yl)-benzonitrile (230 mg, 0.63 mmol) and 3,5-dimethyl isoxazol-4-yl isocyanate. 1-[4-(4-Benzhydryl-piperidin-1-yl)-3-cyano-phenyl]-3-(3,5-dimethyl-isoxazol-4-yl)-urea (255 mg, 80.6% yield) was obtained as a white powder.

LC/MS: r.t. 6.97 min, 506.4 [M+H]

### EXAMPLE 14

### Tetrahydro-furan-3-carboxylic acid [4-(4-benzhydryl-piperidin-1-yl)-3-cyano-phenyl]-amide

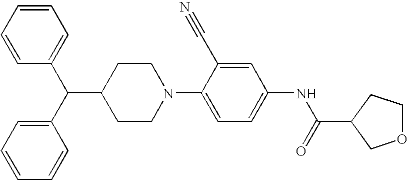

Synthesis analogous to Example 9 with starting with 5-amino-2-(4-benzhydryl-piperidin-1-yl)-benzonitrile (230 mg, 0.63 mmol) and tetrahydro-3-furoic acid. Tetrahydro-furan-3-carboxylic acid [4-(4-benzhydryl-piperidin-1-yl)-3-cyano-phenyl]-amide (192 mg, 76% yield) was obtained as a white powder.

LC/MS: r.t. 6.18 min, 466.4 [M+H]

### EXAMPLE 15

### N-[4-(4-Benzhydryl-piperidin-1-yl)-3-cyano-phenyl]-2-ethyl-butyramide

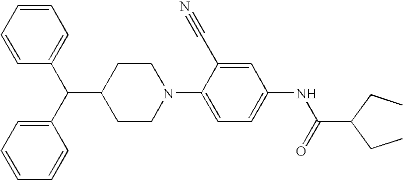

Synthesis analogous to Example 9 with starting with 5-amino-2-(4-benzhydryl-piperidin-1-yl)-benzonitrile (230 mg, 0.63 mmol) and 2-ethyl-butyryl chloride. N-[4-(4-Benzhydryl-piperidin-1-yl)-3-cyano-phenyl]-2-ethyl-butyramide (206 mg, 71% yield) was obtained as a white powder.

LC/MS: r.t. 7.01 min, 466.4 [M+H]

### EXAMPLE 16

### 1-[4-(4-Benzhydryl-piperidin-1-yl)-3-fluoro-phenyl]-3-(3,5-dimethyl-isoxazol-4-yl)-urea

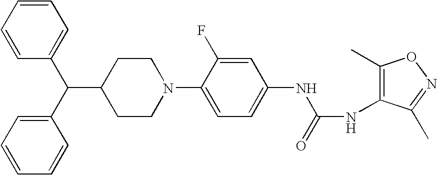

### Step a: 4-Benzhydryl-1-(2-fluoro-4-nitro-phenyl)-piperidine

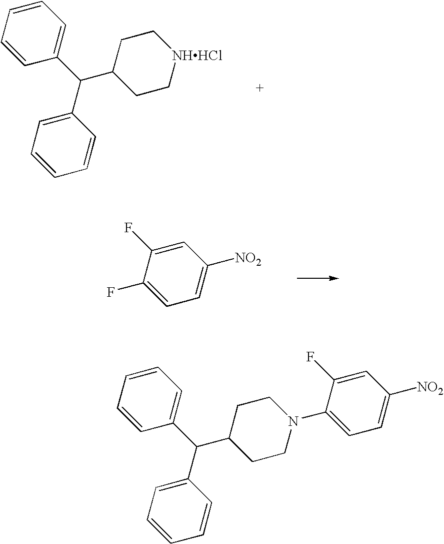

Synthesis analogous to Example 13 step a with starting with 4-benzhydryl-piperidine hydrochloride salt (2.6 g, 9.03 mmol) and 3,4-difluoro-nitrobenzene. 4-Benzhydryl-1-(2-fluoro-4-nitro-phenyl)-piperidine (2.9 g, 82.2% yield) was obtained as a yello-orange powder.

LC/MS: r.t. 6.16 min, 391.3 [M+H] gradient de 50% to 100% B

### Step b: 4-(4-Benzhydryl-piperidin-1-yl)-3-fluoro-phenylamine

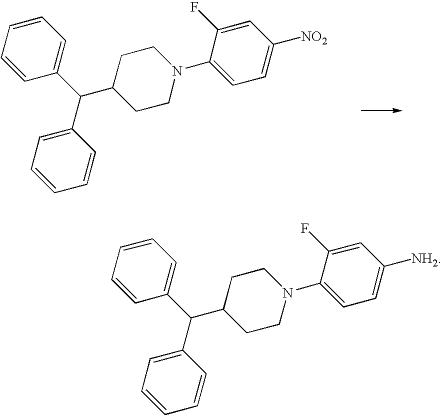

Synthesis analogous to Example 1 step d with starting with 4-benzhydryl-1-(2-fluoro-4-nitro-phenyl)-piperidine (2.8 g, 7.17 mmol). 4-(4-Benzhydryl-piperidin-1-yl)-3-fluoro-phenylamine (2.04 g, 79% yield) was obtained as a yellow powder.

LC/MS: r.t. 6.01 min, 361.2 [M+H]

### Step c: 1-[4-(4-Benzhydryl-piperidin-1-yl)-3-fluoro-phenyl]-3-(3,5-dimethyl-isoxazol-4-yl)-urea

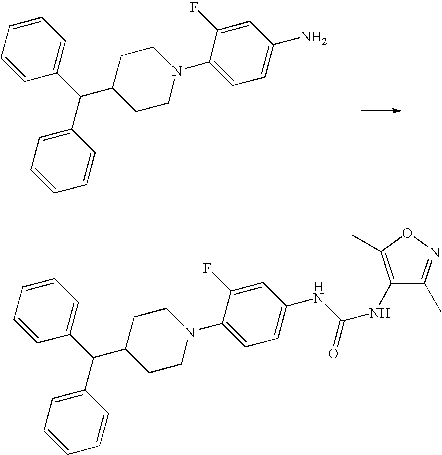

Synthesis analogous to Example 1 step e with starting with 4-(4-benzhydryl-piperidin-1-yl)-3-fluoro-phenylamine (230 mg, 0.64 mmol) and 3,5-dimethyl isoxazol-4-yl isocyanate. 1-[4-(4-Benzhydryl-piperidin-1-yl)-3-fluoro-phenyl]-3-(3,5-dimethyl-isoxazol-4-yl)-urea (182 mg, 57.2% yield) was obtained as a white powder.

LC/MS: r.t. 6.11 min, 499.4 [M+H]

### EXAMPLE 17

### Tetrahydro-furan-3-carboxylic acid [4-(4-benzhydryl-piperidin-1-yl)-3-fluoro-phenyl]-amide

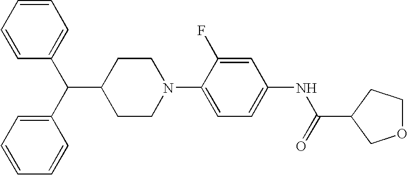

Synthesis analogous to Example 9 with starting with 4-(4-benzhydryl-piperidin-1-yl)-3-fluoro-phenylamine (230 mg, 0.64 mmol) and tetrahydro-3-furoic acid. Tetrahydro-furan-3-carboxylic acid [4-(4-benzhydryl-piperidin-1-yl)-3-fluoro-phenyl]-amide (172 mg, 58.8% yield) was obtained as a white powder.

LC/MS: r.t. 6.82 min, 459.4 [M+H]

### EXAMPLE 18

### N-[4-(4-Benzhydryl-piperidin-1-yl)-3-fluoro-phenyl]-2-ethyl-butyramide

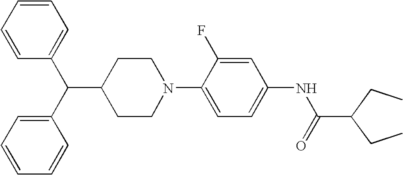

Synthesis analogous to Example 9 with starting with 4-(4-benzhydryl-piperidin-1-yl)-3-fluoro-phenylamine (200 mg, 0.56 mmol) and 2-ethyl-butyryl chloride. N-[4-(4-Benzhydryl-piperidin-1-yl)-3-fluoro-phenyl]-2-ethyl-butyramide (152 mg, 59.7% yield) was obtained as a white powder.

LC/MS: r.t. 6.83 min, 459.4 [M+H]

### EXAMPLE 19

### 5-(4-Benzhydryl-piperazin-1-yl)-2-(1-ethyl-propyl)-6-fluoro-1H-benzoimidazole

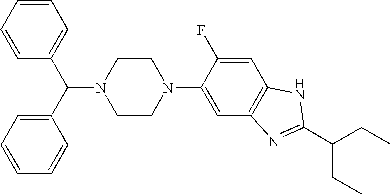

### Step a: 5-(4-Benzhydryl-piperazin-1-yl)-4-fluoro-2-nitro-phenylamine

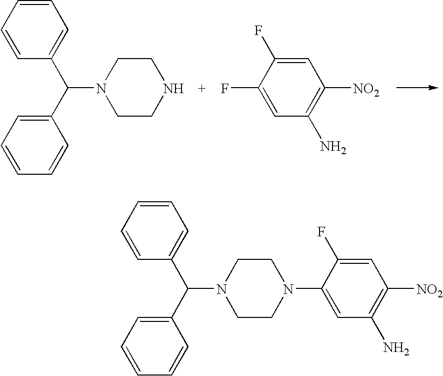

A mixture of mono-benzhydrylpiperazine (2.6 g, 10.3 mmol), 4,5-difluoro-2-nitroaniline (1.97 g, 1.1 eq) and potassium carbonate (2.16 g, 1.5 eq) in DMF (25 mL) was stirred at 50° C. for 2 hours. The mixture was quenched with water and the pH of the aqueous was adjusted to pH 9-10 by addition of NaOH 2N. The aqueous phase was extracted with AcOEt. The organic phases were combined, washed with brine, dried over sodium sulfate and concentrated in vacuo to afford a crude orange oil. This oil was sonicated in Et₂O/Hexanes and the resulting precipitate was filtered off and washed with Hexanes. After high-vacuum drying, 5-(4-benzhydryl-piperazin-1-yl)-4-fluoro-2-nitro-phenylamine (3.3 g, 78.8% yield) was obtained as an orange powder.

LC/MS: r.t. 4.82 min, 407.2 [M+H]

### Step b. N-[5-(4-Benzhydryl-piperazin-1-yl)-4-fluoro-2-nitro-phenyl]-2-ethyl-butyramide

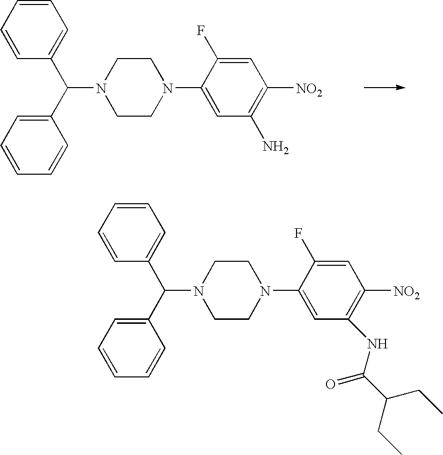

Synthesis analogous to Example 9 with starting with 5-(4-benzhydryl-piperazin-1-yl)-4-fluoro-2-nitro-phenylamine (500 mg, 1.23 mmol) and 2-ethyl-butyryl chloride. N-[5-(4-Benzhydryl-piperazin-1-yl)-4-fluoro-2-nitro-phenyl]-2-ethyl-butyramide (450 mg, 72.5% yield) was obtained as a yellow powder.

LC/MS: r.t. 5.52 min, 505.4 [M+H]

### Step c: 5-(4-Benzhydryl-piperazin-1-yl)-2-(1-ethyl-propyl)-6-fluoro-1H-benzoimidazole

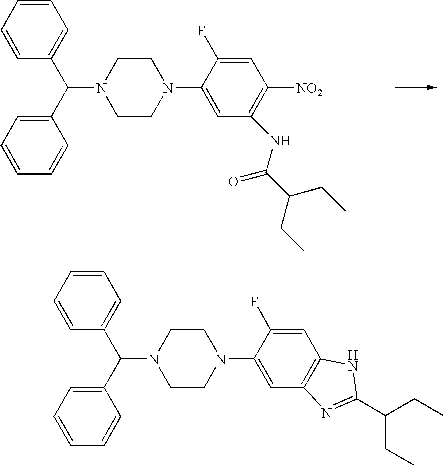

A mixture of N-[5-(4-benzhydryl-piperazin-1-yl)-4-fluoro-2-nitro-phenyl]-2-ethyl-butyramide (200 mg, 0.4 mmol) and tin chloride hydrate (537 mg, 6 eq) in EtOH (20 mL) was refluxed for 48 hours. The solvent was removed in vacuo and the crude was taken in water water and the pH of the aqueous was adjusted to pH 9-10 by addition of NaOH 2N. The aqueous phase was extracted with AcOEt. The organic phases were combined, washed with brine, dried over sodium sulfate and concentrated in vacuo to afford a crude brown solid. The crude material was purified by flash chromatography on silica gel using DCM/MeOH (100/0 to 90/10) as solvent system. From the purification, 5-(4-benzhydryl-piperazin-1-yl)-2-(1-ethyl-propyl)-6-fluoro-1H-benzoimidazole (60 mg, 33.2% yield) was obtained as a beige powder.

LC/MS: r.t. 4.22 min, 457.4 [M+H]

### EXAMPLE 20

### 5-(4-Benzhydryl-piperazin-1-yl)-6-fluoro-2-(tetrahydro-furan-3-yl)-1H-benzoimidazole

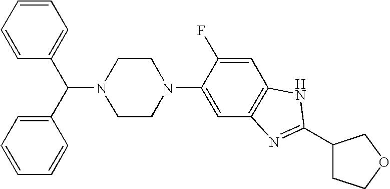

Synthesis analogous to Example 19 step b and c, with starting with 5-(4-benzhydryl-piperazin-1-yl)-4-fluoro-2-nitro-phenylamine and tetrahydro-3-furoic acid 5-(4-Benzhydryl-piperazin-1-yl)-6-fluoro-2-(tetrahydro-furan-3-yl)-1H-benzoimidazole (67 mg, 41% yield) was obtained as a beige powder.

LC/MS: r.t. 3.88 min, 457.4 [M+H]

### EXAMPLE 21

### 5-(4-Benzhydryl-piperazin-1-yl)-2-(3,5-dimethyl-isoxazol-4-yl)-6-fluoro-1H-benzoimidazole

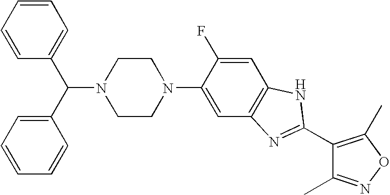

Synthesis analogous to Example 19 step b and c, with starting with 5-(4-benzhydryl-piperazin-1-yl)-4-fluoro-2-nitro-phenylamine and 3,5-dimethyl-isoxazole-4-carboxylic acid. 5-(4-Benzhydryl-piperazin-1-yl)-6-fluoro-2-(tetrahydro-furan-3-yl)-1H-benzoimidazole (48 mg, 41% yield) was obtained as a white powder.

LC/MS: r.t. 4.33 min, 482.4 [M+H]

### EXAMPLE 22

### [5-(4-Benzhydryl-piperazin-1-yl)-6-fluoro-1H-benzoimidazol-2-yl]-(3,5-dimethyl-isoxazol-4-yl)-amine triluoroacetate salt

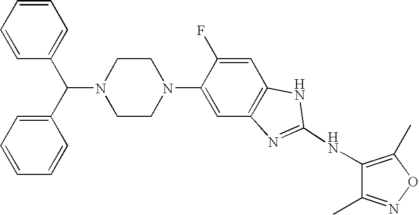

### Step a: 4-(4-Benzhydryl-piperazin-1-yl)-5-fluoro-benzene-1,2-diamine

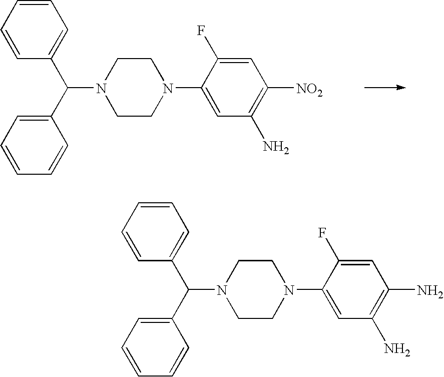

Synthesis analogous to Example 1 step d with starting with 5-(4-benzhydryl-piperazin-1-yl)-4-fluoro-2-nitro-phenylamine (800 mg, 1.97 mmol). 4-(4-Benzhydryl-piperazin-1-yl)-5-fluoro-benzene-1,2-diamine (495 mg, 66.8% yield) was obtained as a beige powder.

LC/MS: r.t. 3.69 min, 377.3 [M+H]

### Step b: 1-[2-Amino-4-(4-benzhydryl-piperazin-1-yl)-5-fluoro-phenyl]-3-(3,5-dimethyl-isoxazol-4-yl)-thiourea

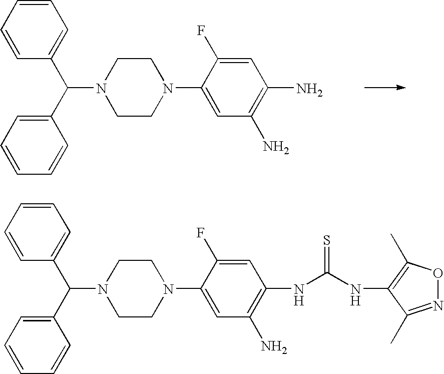

To a solution of 4-(4-benzhydryl-piperazin-1-yl)-5-fluoro-benzene-1,2-diamine (240 mg, 0.64 mmol) in acetonitrile (10 mL), a solution of 3,5-dimethyl-4-isoxazolylisothiocyanate (98.3 mg, 1.0 eq) in 1 mL of acetonitrile was added drop-wise at 0° C. Then, the mixture was stirred at RT for 6 hours. The sovent was removed in vacuo and the mixture was taken in AcOEt. The organic phase was washed with NaOH 2N, water and brine, dried over sodium sulfate and concentrated in vacuo to afford 320 mg of an orange-red crude oil. The crude material was purified by flash chromatography on silica gel using Hexanes/AcOEt (100/0 to 0/100) as solvent system. From the purification, 1-[2-amino-4-(4-benzhydryl-piperazin-1-yl)-5-fluoro-phenyl]-3-(3,5-dimethyl-isoxazol-4-yl)-thiourea (226 mg, 66.8% yield) was obtained as a beige powder.

LC/MS: r.t. 4.33 min, 531.4 [M+H]

### Step c: [5-(4-Benzhydryl-piperazin-1-yl)-6-fluoro-1H-benzoimidazol-2-yl]-(3,5-dimethyl-isoxazol-4-yl)-amine triluoroacetate salt

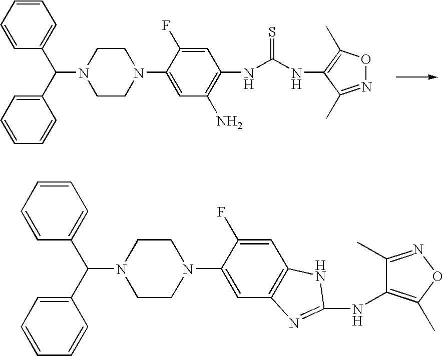

A mixture of 1-[2-amino-4-(4-benzhydryl-piperazin-1-yl)-5-fluoro-phenyl]-3-(3,5-dimethyl-isoxazol-4-yl)-thiourea (226 mg, 0.32 mmol), HgO (184 mg, 2.66 eq), sulfur (2.73 mg, 0.26 eq) in EtOH was refluxed for 5 hours. Then, 1 mL of triethylamine was added and the mixture was refluxed of 16 additional hours. The mixture was cooled down and filtered through a pad of celite. The solvent was concentrate din vacuo to afford a red-brown oil. The crude product was purified by preparative HPLC (Gilson Trilution LC, Column:SunFire C18, 30×100 mm, 5 um, Eluent:Water (+0.1% TFA)/acetonitrile (+0.1% TFA) from 85/15 to 65/35 in 16 min; flow 50 mL/min)

After concentration of the fractions and lyophilization, [5-(4-Benzhydryl-piperazin-1-yl)-6-fluoro-1H-benzoimidazol-2-yl]-(3,5-dimethyl-isoxazol-4-yl)-amine triluoroacetate salt (20 mg, 10.3% yield) was obtained as a beige powder.

LC/MS: r.t. 4.09 min, 497.4 [M+H]

### EXAMPLE 23

### 5-(4-Benzhydryl-piperazin-1-yl)-6-fluoro-1H-benzoimidazol-2-yl]-(1-ethyl-propyl)-amine triluoroacetate salt

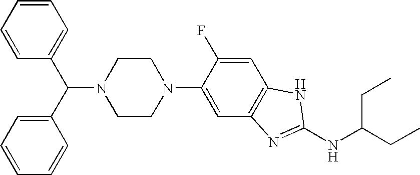

Synthesis analogous to Example 22 step b and c with starting with 4-(4-benzhydryl-piperazin-1-yl)-5-fluoro-benzene-1,2-diamine (240 mg, 0.64 mmol) and 3-pentyl isothiocyanate. 5-(4-Benzhydryl-piperazin-1-yl)-6-fluoro-1H-benzoimidazol-2-yl]-(1-ethyl-propyl)-amine triluoroacetate salt (31.9 mg, 11.8% yield) was obtained as brown powder.

LC/MS: r.t 3.94 min, 472.4 [M+H]

### EXAMPLE 24

### N-{4-[4-(Di-pyridin-2-yl-methyl)-piperidin-1-yl]-3-fluoro-phenyl}-2-ethyl-butyramide

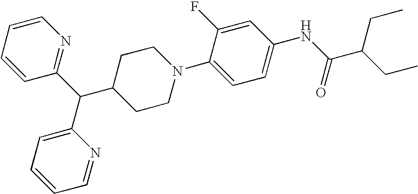

### Step a: 4-(Hydroxy-di-pyridin-2-yl-methyl)-piperidine-1-carboxylic acid tert-butyl ester

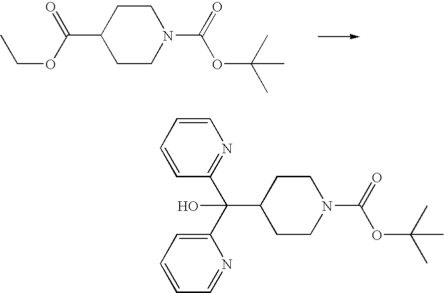

At −70° C., ⁿBuLi (1.6 M in Hexanes, 99 mL, 2.5 eq) was added drop-wise to a solution of 2-bromopyridine (15.4 mL, 2.5 eq) in THF (50 mL). The mixture was stirred at −70° C. for hour prior addition of a solution of piperidine-1,4-dicarboxylic acid 1-tert-butyl ester 4-ethyl ester (16.3 g, 63.3 mmol) in THF. The mixture was kept at −70° C. for 1 hour before to be allowed to reach RT. Then, the mixture was refluxed for 3 days. The mixture was poured into a saturated aqueous solution of NH₄Cl. The aqueous phase was extracted with AcOEt. The organic phases were combined, washed with water, NaOH 2N and brine, dried over sodium sulfate and concentrated in vacuo to afford 28 g of a crude brown oil. The crude product was purified by flash chromatography on silica gel using Hexanes/AcOEt (100/0 to 60/40) as solvent system. From the purification, 4-(hydroxy-di-pyridin-2-yl-methyl)-piperidine-1-carboxylic acid tert-butyl ester (9.4 g, 40% yield) was isolated as an orange oil.

LC/MS: r.t. 4.02 min, 370.1 [M+H]

### Step b: Piperidin-4-yl-di-pyridin-2-yl-methanol

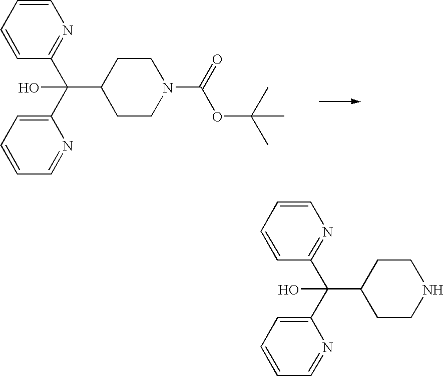

A mixture of 4-(hydroxy-di-pyridin-2-yl-methyl)-piperidine-1-carboxylic acid tert-butyl ester (9.4 g, 25.4 mmol) in 10% TFA/DCM was stirred at RT for 40 hours. The solvents were removed in vacuo and water was added. The pH was adjusted to pH 9-10 by addition of NaOH 30%. The aqueous phase was extracted with DCM. The organic phases were combined, washed with water, dried over sodium sulfate and concentrated in vacuo to afford 6 g of a crude brown oil. The crude product was purified by flash chromatography on silica gel using DCM/MeOH+10% NH₄OH (100 to 80/20) as solvent system. From the purification, piperidin-4-yl-di-pyridin-2-yl-methanol (4.13 g, 60% yield) was isolated as a yellow oil.

LC/MS: r.t. 0.54 min, 270 [M+H]

### Step c: Piperidin-4-yl-di-pyridin-2-yl-methane

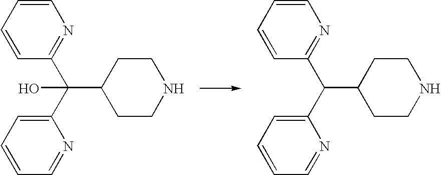

A mixture of piperidin-4-yl-di-pyridin-2-yl-methanol (3.0 g, 11.1 mmol) in SOCl₂ (16.3 mL) was stirred at RT for 20 hours. The solvent was removed in vacuo, the crude was diluted in AcOH and zinc powder (2.9 g, 4 eq) was added. The resulting mixture was stirred at 100° C. for 3 days. The solvent was removed in vacuo and water was added. The pH was adjusted to pH 9-10 by addition of NaOH 2N. The insoluble material was removed by filtration and the aqueous phase was extracted with AcOEt. The organic phases were combined, washed with water, dried over sodium sulfate and concentrated in vacuo to afford 1.94 g (69% yield) of piperidin-4-yl-di-pyridin-2-yl-methane as a brown oil which will be used without further purification.

LC/MS: r.t. 0.54 min, 254 [M+H]

### Step d: 4-(Di-pyridin-2-yl-methyl)-1-(2-fluoro-4-nitro-phenyl)-piperidine

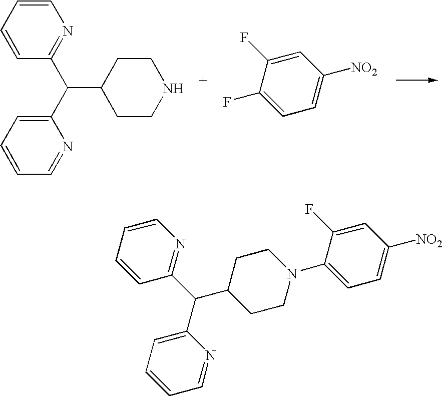

A mixture of piperidin-4-yl-di-pyridin-2-yl-methane (1.16 g, 4.58 mmol), 3,4-difluoronitrobenzene (0.80 g, 1.1 eq) and potassium carbonate (0.94 g, 1.5 eq) in DMF (50 mL) was stirred at 50° C. for 2 hours. The mixture was poured into water and the aqueous phase was extracted with AcOEt. The organic phases were combined, washed with water, dried over sodium sulfate and concentrated in vacuo to afford 2.36 g of a crude yellow oil. The crude product was purified by flash chromatography on silica gel using DCM/MeOH (100 to 95/5) as solvent system. From the purification, 4-(di-pyridin-2-yl-methyl)-1-(2-fluoro-4-nitro-phenyl)-piperidine (1.17 g, 65% yield) was isolated as a yellow oil.

LC/MS: r.t. 4.29 min, 393.1 [M+H]

### Step e: 4-[4-(Di-pyridin-2-yl-methyl)-piperidin-1-yl]-3-fluoro-phenylamine

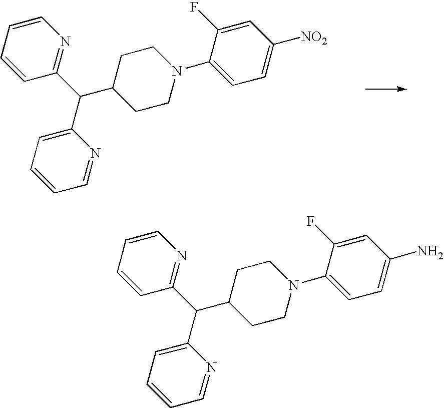

A mixture of 4-(di-pyridin-2-yl-methyl)-1-(2-fluoro-4-nitro-phenyl)-piperidine (1.17 g, 2.98 mmol) and fin chloride dihydrate (3.36 g, 5 eq) in EtOH was refluxed for 2 hours. The solvent was removed in vacuo and water was added. The pH was adjusted to pH 9-10 by addition of NaOH 2N. The aqueous phase was extracted with AcOEt. The organic phases were combined, washed with water, dried over sodium sulfate and concentrated in vacuo to afford 0.9 g of a crude yellow oil. The crude product was purified by flash chromatography on silica gel using DCM/MeOH (100 to 90/10) as solvent system. From the purification, 4-[4-(di-pyridin-2-yl-methyl)-piperidin-1-yl]-3-fluoro-phenylamine (0.55 g, 51% yield) was isolated as a beige solid.

LC/MS: r.t. 2.93 min, 363.1 [M+H]

### Step f: N-{4-[4-(Di-pyridin-2-yl-methyl)-piperidin-1-yl]-3-fluoro-phenyl}-2-ethyl-butyramide

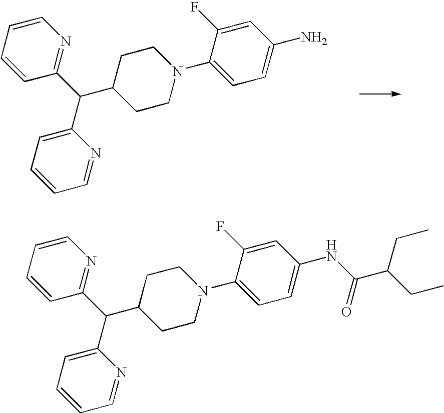

To a solution of 4-[4-(di-pyridin-2-yl-methyl)-piperidin-1-yl]-3-fluoro-phenylamine (150 mg, 0.4 mmol) and TEA (0.087 mL, 1.5 eq) in DCE, 2-ethyl-butyryl chloride (0.068 mL, 1.2 eq) was added. The resulting mixture was stirred at RT for 1 hour. The organic phase was washed with NaOH 2N, dried over sodium sulfate and concentrated in vacuo to a crude beige solid (185 mg). The crude product was purified by flash chromatography on silica gel using DCM/MeOH (100 to 90/10) as solvent system. From the purification, N-{4-[4-(Di-pyridin-2-yl-methyl)-piperidin-1-yl]-3-fluoro-phenyl}-2-ethyl-butyramide (0.085 g, 45% yield) was isolated as a light yellow powder.

LC/MS: r.t. 4.03 min, 461.1 [M+H]

### EXAMPLE 25

### 2-Ethyl-N-{3-fluoro-4-[4-((R)-pyridin-2-yl-tetrahydro-furan-2-yl-methyl)-piperazin-1-yl]-phenyl}-butyramide

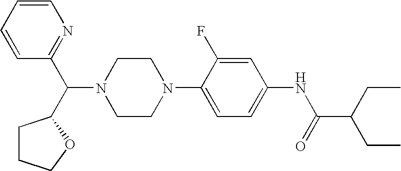

### Step a: Pyridin-2-yl-(R)-tetrahydro-furan-2-yl-methanone

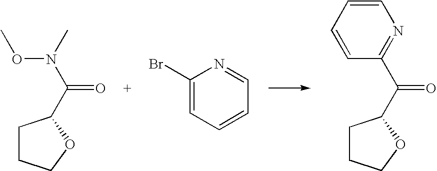

At −78° C., to a solution of 2-bromopyridine (2 mL, 1.2 eq) in THF (50 mL), ⁿBuLi 1.6 M in hexanes (12.8 mL, 1.2 eq) was added drop-wise. The mixture was kept for one hour at −78° C. before addition of a solution of (R)-tetrahydro-furan-2-carboxylic acid methoxy-methyl-amide (2.72 g, 17.1 mmol) in THF. At the end of the addition, the mixture was allowed to reach RT and was then stirred at 60° C. for 2 hours. The mixture was then cooled at 0° C. and diethyl ether (80 mL) and saturated aqueous NH₄Cl (10 mL) were added. The organic phase was separated, dried over sodium sulfate and concentrated in vacuo to afford pyridin-2-yl-(R)-tetrahydro-furan-2-yl-methanone (2.92 g, 96% yield) as a brown oil which will be used without further purification.

LC/MS: r.t. 3.0 min, 177.9 [M+H]

### Step b: Pyridin-2-yl-(R)-tetrahydro-furan-2-yl-methanol

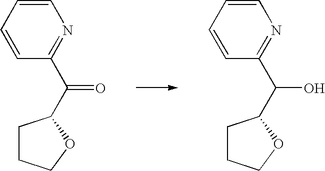

A mixture of pyridin-2-yl-(R)-tetrahydro-furan-2-yl-methanone (2.92 g, 16.5 mmol) and NaBH₄ (623 mg, 1 eq) in MeOH (40 mL) was stirred at RT for 16 hours. Water was added and the MeOH was removed in vacuo. The aqueous phase was extracted with AcOEt. The organic phases were combined, dried over sodium sulfate and concentrated in vacuo to afford pyridin-2-yl-(R)-tetrahydro-furan-2-yl-methanol (2.22 g, 75% yield) which will be used without further purification.

LC/MS: r.t. 0.6 min, 179.9 [M+H]

### Step c: 2-((R)-Chloro-tetrahydro-furan-2-yl-methyl)-pyridine

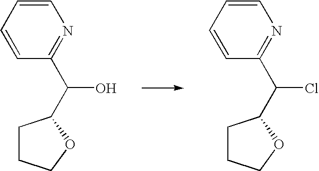

A mixture of pyridin-2-yl-(R)-tetrahydro-furan-2-yl-methanol (2.22 g, 12.4 mmol) in thionyl chloride (2.7 mL) was stirred at RT for 1 hour. Concentration in vacuo led to the 2-((R)-chloro-tetrahydro-furan-2-yl-methyl)-pyridine (2.44 g, 100% yield) as a black paste which will be used without further purification.

LC/MS: r.t. 3.04 min, 197.8/199.8 [M+H]

### Step d: 1-((R)-Pyridin-2-yl-tetrahydro-furan-2-yl-methyl)-piperazine

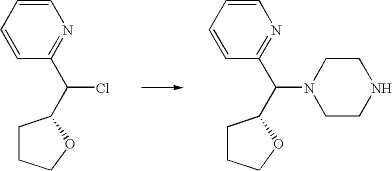

A mixture of 2-((R)-chloro-tetrahydro-furan-2-yl-methyl)-pyridine (925 mg, 4.7 mmol), piperazine (1.6 g, 4 eq), potassium carbonate (650 mg, 1 eq) and KI (777 mg, 1 eq) in DMF (10 mL) was stirred at 130° C. for 16 hours. The mixture was diluted with water and the aqueous phase was extracted with DCM. The organic phases were combined, dried over sodium sulfate and concentrated in vacuo to afford a crude brown oil (2.2 g). The crude product was purified by flash chromatography on silica gel using DCM/MeOH+10% NH₄OH (100/0 to 70/30) as solvent system. From the purification, 1-((R)-pyridin-2-yl-tetrahydro-furan-2-yl-methyl)-piperazine (900 mg, 78% yield) was isolated as a brown oil.

LC/MS: r.t. 1.22 min, 248.0 [M+H]

### Step e: 1-(2-Fluoro-4-nitro-phenyl)-4-((R)-pyridin-2-yl-tetrahydro-furan-2-yl-methyl)-piperazine

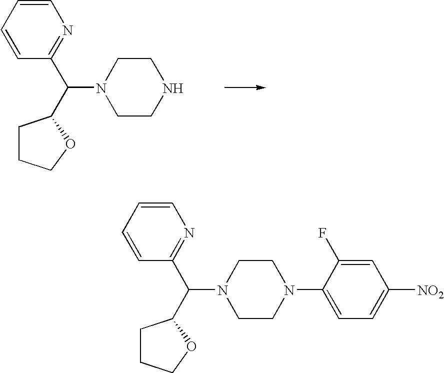

Synthesis analogous to Example 24 step d starting with 1-((R)-pyridin-2-yl-tetrahydro-furan-2-yl-methyl)-piperazine (900 mg, 3.64 mmol). 1-(2-Fluoro-4-nitro-phenyl)-4-((R)-pyridin-2-yl-tetrahydro-furan-2-yl-methyl)-piperazine (670 mg, 48% yield) was obtained as a red-yellow oil.

LC/MS: r.t. 4.04 min, 387.1 [M+H]

### Step f: 3-Fluoro-4-[4-((R)-pyridin-2-yl-tetrahydro-furan-2-yl-methyl)-piperazin-1-yl]-phenylamine

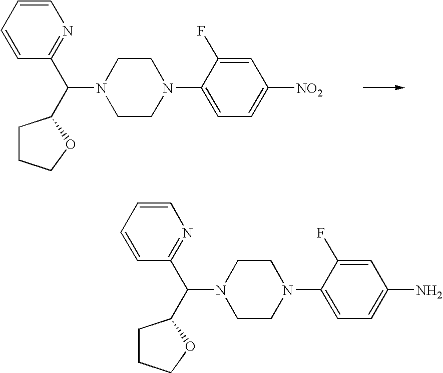

Synthesis analogous to Example 24 step e starting with 1-(2-fluoro-4-nitro-phenyl)-4-((R)-pyridin-2-yl-tetrahydro-furan-2-yl-methyl)-piperazine (670 mg, 1.73 mmol). 3-Fluoro-4-[4-((R)-pyridin-2-yl-tetrahydro-furan-2-yl-methyl)-piperazin-1-yl]-phenylamine (384 mg, 62% yield) was obtained as a light yellow oil.

LC/MS: r.t. 2.33 min, 357.1 [M+H]

### Step g: 2-Ethyl-N-{3-fluoro-4-[4-((R)-pyridin-2-yl-tetrahydro-furan-2-yl-methyl)-piperazin-1-yl]-phenyl}-butyramide

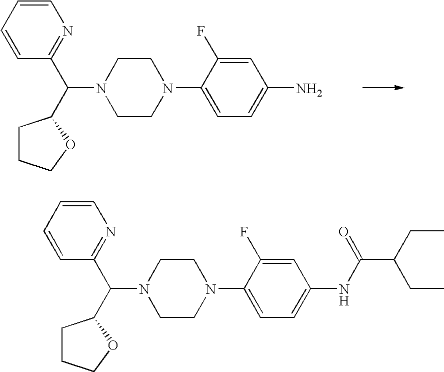

Synthesis analogous to Example 24 step f starting with 3-fluoro-4-[4-((R)-pyridin-2-yl-tetrahydro-furan-2-yl-methyl)-piperazin-1-yl]-phenylamine (150 mg, 0.42 mmol). 2-Ethyl-N-{3-fluoro-4-[4-((R)-pyridin-2-yl-tetrahydro-furan-2-yl-methyl)-piperazin-1-yl]-phenyl}-butyramide (93 mg, 49% yield) was obtained as a white powder.

LC/MS: r.t. 4.26 min, 455.3 [M+H]

Biological Testing:

Antagonistic activity of compounds of the present invention is examined by the Scintillation proximity [³⁵S]GTPγS binding assay as described above (inhibition of 0.5 nM NPY-stimulated [³⁵S]GTPγS binding). The table below represents percentages of inhibition at a concentration of 10 μM.

| | [%] inhibiton |
|---|---|
| Compound | at 10 μM |
| 1 | 95 |
| 2 | 89 |
| 3 | 88 |
| 4 | 89 |
| 5 | 85 |
| 6 | 89 |
| 7 | 87 |
| 8 | 86 |
| 9 | 85 |
| 10 | 85 |
| 11 | 62 |
| 12 | 65 |
| 13 | 91 |
| 14 | 90 |
| 15 | 86 |
| 16 | 90 |
| 17 | 86 |
| 18 | 85 |
| 19 | 82 |
| 20 | 53 |
| 21 | 44 |
| 22 | 71 |
| 23 | 72 |
| 24 | 95 |
| 25 | 90 |

1. A compound of the formula I 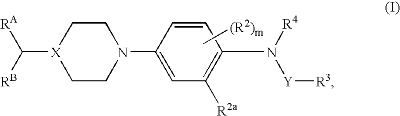
wherein
R^{A} and R^{B} independently represent an optionally substituted aryl group, an optionally substituted cycloalkyl group, an optionally substituted heteroaryl group, an optionally substituted heterocyclyl group;
X represents CH or N;
R² and R^{2a} independently represent hydrogen or a substituent as defined for R¹; or a single bond attached to R⁴;
m represents an integer from 0-3;
Y represents a single bond, —C(O)—, —C(O)N(R⁵)—, —(CH₂)ₚ—, —O—, —N(R⁵)—, —O—C(O)—, —C(O)O—;
or Y represents one of the following groups 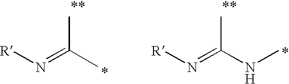
wherein R′ and R^{2a} together represent a single bond, wherein the bond marked * is attached to R³, and wherein the bond marked ** is attached to the nitrogen atom bound to the phenyl ring;
R⁵ represents hydrogen, alkyl, cycloalkyl;
p represents an integer from 0-5;
R³ represents an optionally substituted aryl group, an optionally substituted cycloalkyl group, an optionally substituted heteroaryl group, an optionally substituted heterocyclyl group; an optionally substituted alkyl group;
R⁴ represents hydrogen or a sustituent as defined for R³;
or, in case R² or R^{2a} represents a single bond attached to R⁴, R⁴ represents alkanediyl or alkenediyl, in each case optionally substituted by a substituent as defined for R¹ and in each case optionally interrupted by one or more moieties selected from the group consisting of —O—, ═N—; —N(R⁵)—;
provided in case X represents N and Y represents —C(O)—, R³ does not represent substituted phenyl or substituted indolyl;
in free base form or in acid addition salt form.

2. A compound of the formula IAA 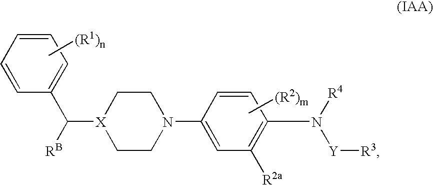
wherein
R^{B} represents an optionally substituted aryl group, an optionally substituted cycloalkyl group, an optionally substituted heteroary group, an optionally substituted heterocyclyl group;
R¹ represents a substituent different from hydrogen;
n represents an integer from 0-5;
X represents CH or N;
R² and R^{2a} independents represent hydrogen or a substituent as defined for R¹; or a single bond attached to R⁴;
m represents an integer from 0-3;
Y represents a single bond, —C(O)—, —C(O)N(R⁵)—, —(CH₂)ₚ—, —O—, —N(R⁵)—, —O—C(O)—, —C(O)O—;
or Y represents one of the following groups 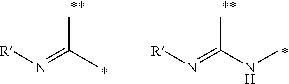
wherein R′ and R^{2a} together represent a single bond, wherein the bond marked * is attached to R³, and wherein the bond marked ** is attached to the nitrogen atom bound to the phenyl ring;
R⁵ represents hydrogen, alkyl, cycloalkyl;
p represents an integer from 0-5;
R³ represents an optionally substituted aryl group, an optionally substituted cycloalkyl group, an optionally substituted heteroaryl group, an optionally substituted heterocyclyl group; an optionally substituted alkyl group;
R⁴ represents hydrogen or a sustituent as defined for R³;
or, in case R² or R^{2a} represents a single bond attached to R⁴, R⁴ represents alkanediyl or alkenediyl, in each case optionally substituted by a substituent as defined for R¹ and in each case optionally interrupted by one or more moieties selected from the group consisting of —O—, ═N—; —N(R⁵)—;
provided in case X represents N and Y represents —C(O)—, R³ does not represent substituted phenyl or substituted indolyl;
in free base form or in acid addition salt form.

3-5. (canceled)

6. A method for the treatment, prevention or delay of progression of a condition, disease or disorder, that can be modulated or is mediated by NPY Y2 receptors, comprising:
administering to a subject in need thereof a therapeutically effective amount of a compound of the formula I′, 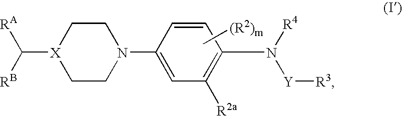
wherein
R^{A} and R^{B} independently represent an optionally substituted aryl group, an optionally substituted cycloalkyl group, an optionally substituted heteroaryl group, an optionally substituted heterocyclyl group;
X represents CH or N;
R² and R^{2a} independently represent hydrogen or a substituent as defined for R¹; or a single bond attached to R⁴;
m represents an integer from 0-3;
Y represents a single bond, —C(O)—, —C(O)N(R⁵)—, —(CH₂)ₚ—, —O—, —N(R⁵)—, —O—C(O)—, —C(O)O—;
or Y represents one of the following groups 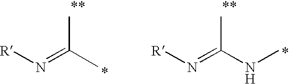
wherein R′ and R^{2a} together represent a single bond, wherein the bondmarked * is attached to R³, and wherein the bond marked ** is attached to the nitrogen atom bound to the phenyl ring;
R⁵ represents hydrogen, alkyl, cycloalkyl;
p represents an integer from 0-5;
R³ represents an optionally substituted aryl group, an optionally substituted cycloalkyl group, an optionally substituted heteroaryl group, an optionally substituted heterocyclyl group; an optionally substituted alkyl group;
R⁴ represents hydrogen or a sustituent as defined for R³;
or, in case R² or R^{2a} represents a single bond attached to R⁴, R⁴ represents alkanediyl or alkenediyl, in each case optionally substituted by a substituent as defined for R¹ and in each case optionally interrupted by one or more moieties selected from the group consisting of —O—, ═N—; —N(R⁵)—;
in free form or in pharmaceutically acceptable salt form.

7. A pharmaceutical composition, comprising:
the compound of the formula I as defined in claim 1, in free form or in pharmaceutically acceptable salt form, as active ingredient, in association with a pharmaceutical carrier or diluent.

8. A combination comprising a therapeutically effective amount of a compound of the formula I as defined in claim 1, in free form or in pharmaceutically acceptable salt form, and a second drug substance, for simultaneous or sequential administration.

9. A pharmaceutical composition, comprising:
the compound of the formula I as defined in claim 2, in free form or in pharmaceutically acceptable salt form, as active ingredient, in association with a pharmaceutical carrier or diluent.
